SEQUENCE LISTING (2 sequences)

SEQ ID NO: 1 - protein, 140 aa, Homo sapiens
  MISC_FEATURE: CST1
MAQYLSTLLLLLATLAVALAWSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQE

SEQ ID NO: 2 - protein, 141 aa, Homo sapiens
  MISC_FEATURE: CST2
MAWPLCTLLLLLATQAVALAWSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

(19) United States
(12) Patent Application Publication
(10) Pub. No.: US 2023/0159659 A1
(43) Pub. Date: May 25, 2023

(54) INHIBITION OF CYSTATINS FOR THE TREATMENT OF CHRONIC RHINOSINUSITIS

(71) Applicants: Massachusetts Eye and Ear Infirmary, Boston, MA (US); Northeastern University, Boston, MA (US)

(72) Inventors: Benjamin S. Bleier, Weston, MA (US); Angela Nocera, Boston, MA (US); Mansoor Amiji, Boston, MA (US)

(21) Appl. No.: 17/261,245

(22) PCT Filed: Jul. 29, 2019
(86) PCT No.: PCT/US2019/043928

§ 371 Date: Jan. 19, 2021

Related U.S. Application Data
(60) Provisional application No. 62/711,288, filed on Jul. 27, 2018.

(51) Int. Cl. C07K 16/38; A61K 45/06; A61K 9/00; A61K 31/573

(52) U.S. Cl. CPC C07K 16/38; A61K 45/06; A61K 9/0024; A61K 31/573

(57) ABSTRACT

Methods and compositions for the treatment of chronic rhinosinusitis in a subject, and more particularly to methods for treating chronic rhinosinusitis with nasal polyps in a subject with cystatin inhibitors.

### CLAIM OF PRIORITY

This application claims the benefit of U.S. Patent Application Ser. No. 62/711,288, filed on Jul. 27, 2018. The entire contents of the foregoing are hereby incorporated by reference.

### SEQUENCE LISTING

The instant application contains a Sequence Listing which has been submitted electronically in ASCII format and is hereby incorporated by reference in its entirety. Said ASCII copy, created on Jun. 24, 2021, is named “Sequence_Listing.txt” and is 3 KB in size.

### TECHNICAL FIELD

This invention relates to treatment of chronic rhinosinusitis in a subject, and more particularly to methods for treating chronic rhinosinusitis with nasal polyps in a subject with cystatin inhibitors.

### BACKGROUND

Paranasal sinuses are four pairs of air-filled cavities connecting to the nasal passage. The paranasal sinuses are named after the cranial bones in which they are located: the frontal sinuses, the maxillary sinuses, the ethmoid sinuses, and the sphenoid sinuses. A membrane lining the paranasal sinuses secretes mucus, which drains into the nasal passage through a small channel in each sinus. Healthy sinuses likely contain healthy commensal bacteria, and the nasal passage normally contains many bacteria that enter through the nostrils as a person breathes.

A number of factors and processes are involved in maintaining healthy sinuses. The mucus secreted by the membrane lining must be fluid but sticky, in order to flow freely yet absorb pollutants and entrap bacteria. It must also contain sufficient amounts of bacteria-fighting substances such as antibodies. Additionally, small hair-like projections called cilia, located in the nostril, must beat in unison to propel mucus outward, in order to expel bacteria and other particles. Moreover, the mucous membranes themselves must be intact, and the sinus passages must be open to allow drainage and the circulation of air through the nasal passage. When one or more of these processes or factors are amiss, causing obstruction of the sinus passage, an infection called sinusitis develops.

Sinusitis is an inflammation of the mucous membrane lining one or more paranasal sinuses. Rhinitis is an inflammation of the mucous membrane lining the nasal passage. Rhinitis and sinusitis usually coexist and are concurrent in most individuals; thus most guidelines and experts now have adopted the term rhinosinusitis (Fokkens et al., Rhinology 2012 March; 50 (Suppl 23): S5).

The symptoms of rhinosinusitis include nasal congestion and obstruction, colored nasal discharge, anterior or posterior nasal drip. Subjects may also experience facial pain or pressure, and in severe cases, suffer a reduction or a loss of smell (Fokkens et al., 2012). There are two different types of rhinosinusitis: acute and chronic. Acute rhinosinusitis is characterized as rhinosinusitis with complete resolution of symptoms within 12 weeks, while chronic rhinosinusitis lasts longer than 12 weeks, and usually involves tissue damage (Fokkens et al., 2012). There are, generally speaking, two types of chronic sinusitis as well: infectious and inflammatory. These can also be characterized as Th1 vs Th2, or neutrophilic vs eosinophilic types. Nasal polyps, abnormal but benign lesions arising from the mucosa of the nasal sinuses or of the nasal cavity, are frequently present in some subjects with chronic rhinosinusitis (a condition termed Chronic Rhinosinusitis with Nasal Polyps (CRSwNP)) based on epidemiologic studies; typically polyps are associated with the more inflammatory type of chronic rhinosinusitis. Often the polyps are benign semitransparent lesions present at the outflow tract of the sinuses. See, e.g., McClay et al., Nasal Polyps, emedicine.medscape.com/article/994274-overview (Dec. 14, 2017); Newton and Ah-See, Ther Clin Risk Manag. 2008 April; 4(2): 507-512.

### SUMMARY

Chronic Rhinosinusitis with Nasal Polyps (CRSwNP) is an eosinophilic, Th2 driven, disease characterized by chronic nasal inflammation. To date there is no cure for this disease. Current therapies seek to reduce the inflammation which occurs as a consequence of the disease including steroids, biologics, and P-glycoprotein inhibitors. Using a proteomic approach the present inventors discovered that Cystatin SN and SA proteins (gene names CST1 and 2) are profoundly overexpressed in CRSwNP, in tissue, in mucus, and in exosomes (See FIG. 1). These findings were confirmed by Western blot (FIG. 2A) and overexpression was localized to the epithelial surface by immunohistochemistry (FIG. 2B). In an effort to characterize the level at which this gene is overexpressed a matched whole transcriptome analysis was then performed, which confirmed that CST1 and 2 mRNA are highly overexpressed in CRSwNP (FIG. 5). As shown herein, exposure to increased levels of both of these proteins induced significant inflammation in polyp explants which is commensurate with bacterial derived toxins which are known to induce Th2 inflammation (FIG. 6). Finally, inhibition of CST protease inhibitory activity alone is also capable of abrogating proinflammatory cytokine secretion (FIGS. 7A-E). The confluence of these findings, namely that Cystatin SN and SA are profoundly overexpressed in CRSwNP, localize to the epithelial surface, and induce significant inflammation suggest that 1) inhibition of protein EXPRESSION and 2) inhibition of protein ACTIVITY, provides a novel therapy to not only treat CRSwNP but perhaps to offer a cure. Thus disclosed herein are, inter alia, methods for treating rhinosinusitis with cystatin inhibitors.

Thus, provided herein are methods for treating or reducing risk of developing chronic rhinosinusitis with polyps in a subject. The methods include identifying a subject having chronic rhinosinusitis with polyps, or who has chronic rhinosinusitis without polyps and is at risk of developing polyps; and administering to the subject an effective amount of a cystatin inhibitor that inhibits Cystatin 1 (CST1) or CST2.

In some embodiments, the subject has chronic rhinosinusitis with polyps.

In some embodiments, the cystatin inhibitor inhibits CST2.

In some embodiments, the cystatin inhibitor is an antibody that binds specifically to CST1 or CST2.

In some embodiments, the cystatin inhibitor is an inhibitory nucleic acid that targets CST1 or CST2.

In some embodiments, the cystatin inhibitor decreases expression of CST1 and/or CST2 in the subject's sinonasal epithelial cells. In some embodiments, the cystatin inhibitor decreases activity of CST1 and/or CST2 in the subject's sinonasal epithelial cells.

In some embodiments, the cystatin inhibitor is administered systemically.

In some embodiments, the cystatin inhibitor is administered locally to the subject's nasal passage and sinuses. In some embodiments, the cystatin inhibitor is delivered to the subject's nasal passage and sinuses by an inhalation device, by flushing, or by spraying. In some embodiments, the cystatin inhibitor is administered to the subject as a cystatin inhibitor eluting implant, stent, or spacer placed in the subject's nasal passage or sinuses. In some embodiments, the cystatin inhibitor eluting implant is bioabsorbable.

In some embodiments, the subject having rhinosinusitis was identified by endoscopy or by computed tomography. In some embodiments, the subject having rhinosinusitis was identified by a method comprising: obtaining a sample comprising nasal mucus derived exosomes; determining a level of one or both of CST1 or CST2 in the sample; comparing the level of CST1 or CST2 to a reference level; and administering an effective amount of a cystatin inhibitor to a subject who has a level of CST1 or CST2 above the reference level. In some embodiments, the subject having rhinosinusitis was identified by a method comprising obtaining a sample comprising nasal mucus derived exosomes, preferably a sample obtained from the posterior nasal cavity; determining a level of one, two or all three biomarkers selected from (i) one or both of CST1 or CST2; (ii) PRDX5 (Peroxiredoxin-5); and (iii) GP6 (Platelet glycoprotein VI) in the sample; comparing the level of the biomarker to a reference level; and administering an effective amount of a cystatin inhibitor to a subject who has a level of any one or more of the biomarkers above the reference level. In some embodiments, whole mucus rather than mucus-derived exosomes is used as a sample.

In some embodiments, the subject having rhinosinusitis was identified by observing the subject's symptoms and duration of symptoms.

In some embodiments, the methods include monitoring the efficacy of the treatment by endoscopy and/or by computed tomography.

In some embodiments, the methods include monitoring the efficacy of the treatment by observing the subject's symptoms and duration of symptoms.

In some embodiments, the methods include surgically removing any nasal polyps present in the subject.

In some embodiments of the methods described herein, the cystatin inhibitor is administered in combination with one or more of a corticosteroid, a decongestant, and an antibiotic.

Also provided herein are kits for use in a method of treating rhinosinusitis in a subject as described herein. The kits can include a pharmaceutical composition comprising an effective amount of a cystatin inhibitor; and a device for delivering the pharmaceutical composition to the subject's nasal passage and sinuses.

In some embodiments, said device delivers the pharmaceutical composition to the subject's nasal passage and sinuses in a liquid, nebulized, or aerosolized form.

In some embodiments, the cystatin inhibitor is administered in combination with one, two, or more of a corticosteroid, a decongestant, an anti-inflammatory agent, and an antibiotic.

In some embodiments, the corticosteroid is selected from dexamethasone, prednisone, prednisolone, triamcinolone, cortisol, budesonide, mometasone, fluticasone, flunisolide, and betamethasone. In some embodiments, the antibiotic is selected from erythromycin, doxycycline, tetracycline, penicillin, beta-lactam, macrolide, fluoroquinolone, cephalosporin, and sulfonamide. In some embodiments, the kit includes a corticosteroid.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A. Histogram of exosomal marker enrichment between the exosome and whole mucus fraction among the CRSwNP and control populations (* p<0.05).

FIG. 1B. 2-dimensional PCA plot demonstrating clustering of exosome (E), tissue (T), and whole mucus (M) proteomes regardless of underlying disease state (P-CRSwNP, C-control). CE and PE clustered in the top left, CM and PM clustered in the bottom left, and CT and PT clustered in the right hand side.

FIG. 2A. Western blot analysis for CRSwNP and control patients of CST 1, PRDX5 and GP6 in tissue and exosomes (GAPDH was used as a housekeeping gene).

FIG. 2B. IHC demonstrating differential expression and localization of CST 1 and 2 to the epithelial cytoplasm in CRSwNP (top row 200×, bottom row 400×).

FIG. 3A. Scatterplots detailing positive Pearson correlations between exosomal CST 1/2 protein with tissue CST1/2 protein, tissue CST1/2 mRNA, and clinical parameters.

FIG. 3B. Correlation matrix demonstrates pearson correlation coefficients (see color bar) between all analyzed variables.

FIG. 4. Histogram (mean and SEM) of exosomal CST protein expression by allergy and AERD status (* p<0.01, ** p=0.01).

FIG. 5. Log fold change of mRNA among all protease inhibitor genes between control (C) and CRSwNP (P) demonstrating not only is the fold change greatest among CST1 but the absolute Fragments Per Kilobase of transcript per Million mapped reads (FPKM) is highest for CST1 within the polyp group.

FIGS. 6A-6C. Stimulation of nasal polyp explants with CST 1 and 2 promote the secretion of TH2 related (e.g., polyp forming) cytokines.

FIG. 7A. Protease assay demonstrating dose dependent increase in protease activity using the canonical protease Papain.

FIG. 7B. Dose dependent abrogation of papain protease activity using recombinant CST confirming in vitro activity of recombinant CST.

FIG. 7C. Inhibition of CST protease inhibitor activity using a monoclonal anti-CST antibody confirming in vitro activity of the CST/anti-CST combination.

FIG. 7D. Recombinant CST2 significantly decreased protease activity.

FIGS. 8A and 8B. Pro-inflammatory cytokine secretion in organotypic nasal polyp explants is induced by exposure to recombinant CST and subsequently abrogated by the addition of the monoclonal anti-CST antibody.

FIG. 9. Prevalence of clinical features among each patient cluster.

FIG. 10. Amino acid sequences of CST 1 (SEQ ID NO:1) and CST 2 (SEQ IS NO:2) proteins with post translational modifications (PTMs) that are present in 50% or more of polyp tissue samples but absent in controls. d, deamidation; a, acetylation; m, methylation.

### DETAILED DESCRIPTION

Chronic Rhinosinusitis impacts more than 30 million Americans resulting in $6.9 to $9.9 billion in annual healthcare expenditures (1,2) and $12.8 billion in productivity costs (3). Using health state utility score comparisons, the disease burden of CRS has been shown to be worse than congestive heart failure, chronic obstructive pulmonary disorder, and even Parkinson's disease (4). The subset of CRS patients with nasal polyps (CRSwNP) have an estimated prevalence between 2-5% (5-7) and represent the most challenging population with respect to disease severity, recurrence after surgery, and lack of effective therapies (6-8). Current state-of-the-art therapies for CRSwNP have targeted the type 2 helper T-cell (Th2) cytokine cascade (9-12) as Th2 skewing has been shown to be a dominant feature of most patients with CRSwNP (13).

Despite the early success of some of these approaches, our current description of the immune pathways underpinning CRSwNP fail to implicate which, if any, environmental etiologic agents are driving these aberrant inflammatory cascades and epithelial barrier dysfunction. Multiple hypotheses have been put forth including bacteria (e.g. Staphylococcus aureus (14) and biofilms (15)), fungus (16), and allergens (17) however none have been definitively linked to the development of nasal polyps and, more specifically, their eradication has not been convincingly shown to reverse the disease process. Moreover several puzzling and seemingly disparate clinical features are evident in CRSwNP which, if unified, could point to a common explanatory framework. These include the significant overlap between allergic rhinitis and CRSwNP with regards to epidemiology and immunopathophysiology (18), the general localization of nasal polyps to the sinus mucosa with relative sparing of the inferior turbinate, the relatively late mean onset of nasal polyps in the early 40s (19), and the similarity between the immune response seen in CRSwNP to that of a parasitic infection (20).

Studies examining the proteome of mucus derived exosomes identified Cystatin SN and SA (gene names CST 1 and 2) as being significantly overexpressed in CRSwNP relative to control. Cystatins constitute a large group of evolutionary related proteins acting as protease inhibitors on papain like proteases belonging to the enzyme family Cl. The type 2 cystatins C, D, E/M, F, S, SN, and SA are characterized by two conserved disulfide bridges and the presence of a signal peptide for extracellular targeting. The purpose of these cystatins are to control cysteine proteases which are widely expressed proteolytic enzymes within allergens (21), viruses, and bacteria and have established roles in inflammatory tissue destruction as well as tissue remodeling (22,23). Cystatins have also been shown to have direct immunomodulatory (24), antimicrobial (24,25), and anti-viral properties (26). CST1 expression has been previously studied in the context of seasonal allergic rhinitis (SAR) and was found to be transiently upregulated in season by both Ndika et al (27) and Imoto et al (22).

Herein, the pattern and degree of epithelial cystatin expression was examined in subjects having chronic sinusitis (CRS) with or without nasal polyposis (NP) and in control subjects. The data presented herein show that the expression of Cystatin is negligible in healthy sinus mucosa, but is significantly elevated in the epithelial layer of sinus mucosa and in exosomes of subjects having CRS with or without NP relative to other non-diseased sinonasal subsites.

The sinonasal epithelium functions as a barrier organ against the external environment and is endowed with an array of innate and adaptive immunologic mechanisms to combat extrinsic pathogens. It has been suggested that sinonasal epithelial cells may function as primary actors in the initiation and maintenance of chronic sinonasal inflammation through the elaboration of an array of cytokines and subsequent recruitment of professional immune cells (Reh et al., Am. J Rhinol. Allergy. 2010; 24(2):105-9). While these studies suggest that epithelial cells are capable of orchestrating an innate immune response, the post-translational mechanisms governing non-canonical cytokine secretion at the cellular level are not fully understood.

The findings of tonic CST 1 and 2 overexpression in CRSwNP exosomes, transient upregulation during SAR exacerbations, and the established immunomodulatory role of cystatins led us to hypothesize that CST 1 and 2 may play a significant role in the etiopathogensis of CRSwNP. The present study utilized an integrated poly-omics approach to study the genetics, expression, and function of CST 1 and 2 in CRSwNP.

Integrated poly-omics refers to a biological analyses approach where multiple omics data sets may be combined through bioinformatics approaches to identify biomarkers and unravel complex geno-pheno-envirotype relationships even with low sample numbers (28,29). This type of approach is well-suited to identify novel disease pathways in rhinosinusitis which fall outside of the traditional innate/adaptive immunity investigative regimes.

Although types 1 and 2 cystatins display considerable differences in amino acid sequence, their tertiary structures are conserved and exhibit a ‘cystatin fold’ that is formed by five stranded anti-parallel b-pleated sheet wrapped around a five-turn a-helix (30).

Ndika et al (27) performed a quantitiative proteomic approach on nasal epithelial brushings from control and seasonal allergic rhinitis (SAR) patients both during and after the pollen season. They found that CST1, which protects against the protease activity of allergens was differentially abundant between controls and SAR during the pollen season and then down regulated when out of season. These findings echoed those of Imoto et al (22) who used a microarray analysis to show that CST1 mRNA was one of 32 genes upregulated in patients exposed to Japanese cedar (Cryptomeria japonica) pollen during exposure. This group also found that CST 1 inhibited histamine release from sensitized basophils and suggested that the role of CST 1 was to inactivate allergen associated proteases. Additionally, Giovannini-Chami et al. (31) found that the transcriptome of pediatric nasal epithelial cells in dust-mite allergic patients demonstrated overexpression of CST 1. CST 1 was also strongly induced in vitro by stimulation with IL-4 and 13.

Poole et al. (32) studied the transcriptome of nasal and lower airway epithelial cells in an effort to distinguish subphenotypes of asthma. They found that IL-13 expression was able to differentiate the more severe Th2 high phenotype from Th2 low patients and that CST 1 expression was one of the most highly correlated genes to IL-13.

Parasitic nematodes express cystatins which function to inhibit host cysteine proteases involved in antigen processing and presentation. These cystatins have also been shown to upregulate IL-10 by macrophages leading to a downregulation of costimulatory molecules and Th2 skewing which promotes a favorable environment for parasitic work growth (33). These findings were supported by Schnoeller et al (34) who also found that filarial cystatins drive macrophage derived IL-10 production. This function drives the hypothesis that helminthic infections antagonize the dominant Th1 response seen in autoimmune disease (20).

Methods of Treating Chronic Rhinosinusitis, e.g., CRSwNP

In some embodiments, a subject with rhinosinusitis is treated with a Cystatin inhibitor (i.e., an inhibitor of CST1, CST2, or both CST1 and 2) in an amount sufficient to inhibit Cystatin function. In some embodiments, the subject has polyps (i.e., CRSwNP), and the treatment is sufficient to reduce the size, growth rate, number, or development of additional polyps, or to reduce the symptoms of CRSwNP. In some embodiments, the subject has CRS without polyps, and the treatment is sufficient to delay or reduce the risk of development of polyps.

In other embodiments, a subject with rhinosinusitis is treated with a Cystatin inhibitor in an amount sufficient to decrease Cystatin expression in the subject's sinonasal epithelial cells, either transcriptionally or posttranscriptionally. Levels in exosomes can be used as a proxy for levels in the epithelial cells.

In some embodiments, the Cystatin inhibitor is administered systemically, e.g., orally, intravenously, intradermally, or subcutaneously. In other embodiments, the Cystatin inhibitor is administered locally to the subject's nasal passage and sinuses by an inhalation device, by flushing, or by spraying. In some embodiments, a subject with rhinosinusitis is treated with nasal drops or sprays comprising an effective amount of a Cystatin inhibitor. An effective amount of the Cystatin inhibitor can be delivered to the subject's nasal passage and sinuses in a liquid form by flushing or spraying or irrigation, or by intranasal drug eluting stent, spacer, or implant, e.g., a submucosal implant (see, e.g., WO 2009/009276). An effective amount of a Cystatin inhibitor can also be delivered to the nasal passage and sinuses of a subject with rhinosinusitis in an aerosolized form by an inhalation device, such as a nebulizer, an inhaler, or an OptiNose.

In some embodiments, a subject with rhinosinusitis is treated with a Cystatin inhibitor in combination with other conventional treatments, e.g., drugs such as corticosteroids and/or antibiotics, to potentiate the effect of treatment. For example, Cystatin inhibitors may be used in combination with a corticosteroid selected from dexamethasone, prednisolone, triamcinolone, cortisol, prednisone, budesonide, mometasone, fluticasone, flunisolide, and betamethasone. In some embodiments, Cystatin inhibitors are used in combination with an antibiotic selected from macrolides, e.g., erythromycin; penicillins, e.g., amoxicillin, beta-lactam, ampicillin; tetracyclines, e.g., doxycycline, tetracycline; sulfonamides, e.g. mafenide, sulfacetamide; fluoroquinolones; and cephalosporins, e.g., ceftaroline fosamil, ceftobiprole. In some embodiments, Cystatin inhibitors are used in combination with a decongestant, e.g., pseudoephedrine, phenylephrine, propylhexedrine, ephedrine, levmetamfetamine, L-desoxyephedrine, naphazoline, xylometazoline, or oxymetazoline.

In some embodiments, Cystatin inhibitors are used in combination with an inhibitor of inflammation, e.g., a small molecule or antibody inhibitor of inflammation, e.g., a pGP inhibitor (e.g., as known in the art or described in WO2014106021, WO2017/173509, WO2017/123933, US2018/0185366, US2017/012844, US2017/0348384, US20150231069; see also Leopoldo et al. (2019) Expert Opinion on Therapeutic Patents, 29:6, 455-461; specific examples include Tariquidar, MC18, Elacridar Zosuquidar, Laniquidar, C-4 Calbiochem, Verapamil, or Desmethylloperamide); a Prostaglandin D2 Receptor 2 (DP2) inhibitor (e.g., fevipiprant, setipiprant, ADC-3680, AZD-1981, MK-1029, MK-7246, OC-459, 00000459, QAV-680, and TM30089; Ramatroban and vidupiprant are non-selective antagonists); anti-inflammatory antibodies targeting IL-5, IL-4, IL-13, and/or IgE, e.g., dupilumab, omalizumab, mepolizumab, and/or benralizumab.

In some embodiments, Cystatin inhibitors are used in combination with two or more of a corticosteroid, an antibiotic, a decongestant, and/or a small molecule or antibody inhibitor of inflammation.

In some embodiments one or more of these agents are used in place of the cystatin inhibitor in a method of treating a subject identified using a method described herein.

In some embodiments, when a subject with rhinosinusitis has nasal polyps, surgical removal of such nasal polyps can be performed in addition to administration of a Cystatin inhibitor to the subject. Thus, a subject with rhinosinusitis may undergo both surgery and treatment with a Cystatin inhibitor.

In some embodiments, a subject continues to experience symptoms of chronic sinusitis after a sinus surgery, and a Cystatin inhibitor-eluting implant, stent, or spacer is used to maintain sinus patency in the subject. During the sinus surgery, a Cystatin inhibitor eluting device is implanted, e.g., in the ostia of the paranasal sinuses to prop open the ostia while locally eluting a Cystatin inhibitor to reduce inflammation of the sinonasal epithelium after the surgery. The Cystatin inhibitor eluting device can be made from bioabsorbable material so that the implant will be absorbed within a short period of time after the implantation and no surgical removal of the implant is necessary. The Cystatin inhibitor eluting device can be in the form of solid, semisolid, gel, polymer, or particle. The implant can also be placed submucosally. In some embodiments, the Cystatin inhibitor eluting device is a bioabsorbable gel such as an alginate gel (e.g., sodium alginate), a cellulose-based gel (e.g., carboxymethyl cellulose or carboxyethyl cellulose), or a chitosan-based gel (e.g., chitosan glycerophosphate; see, e.g., Bleier et al., Am J Rhinol Allergy 23, 76-79, 2009). Spacers, e.g., middle meatal spacers, which can be adapted for use in the present methods and devices are known, including the SINU-FOAM middle meatal spacer described in Rudmik et al., Int Forum Allergy Rhinol. 2012 May-June; 2(3):248-51. These devices can also be used, e.g., before surgery or in a subject who will not or does not plan to undergo surgery. The implantation can be done, e.g., in a surgical suite such as an operating room or in the clinic.

In some embodiments, a tissue sample, e.g., a sinus mucosal biopsy sample, e.g., comprising sinus tissue, mucus, and/or mucus derived exosomes, can be obtained from a subject having rhinosinusitis and one or more tests can be performed on these biopsy samples to assist in selecting a therapy for the subject. For example, levels of Cystatin, PG6, or PRDX5 expression in the nasal epithelium can be determined using methods known in the art, e.g., quantitative fluorescent immunohistochemistry; levels in whole mucus or mucus derived exosomes can also be determined as described herein. When a Cystatin, PG6, or PRDX5 expression level, e.g., in whole mucus, exosomes, or in the nasal epithelium, is determined to be above a threshold (i.e., a reference level), a therapy comprising a treatment described herein, e.g., a Cystatin inhibitor, as described herein can be selected to treat rhinosinusitis in the subject.

In some embodiments, sinus mucosal biopsy samples from a subject having rhinosinusitis and the average number of eosinophils per high powered field can be calculated, e.g., using light microscopy and staining with hematoxylin and eosin. As demonstrated herein, Cystatin expression levels correlate with tissue eosinophilia, thus high levels of tissue eosinpophelia (i.e., levels above a reference level) can be used as a proxy for high levels of Cystatin expression. A therapy as described herein comprising administration of a Cystatin inhibitor can be selected to treat rhinosinusitis in the subject when the average number of eosinophils per high powered field is determined to be above a threshold (i.e., reference level).

In some embodiments, computed tomography (CT) can be performed to score osteitis in a subject having rhinosinusitis. For example, a Kennedy Osteitis Score (KOS) (Lee J T, Kennedy D W, Palmer J N, Am J Rhinol 20:278-282, 2006) or Global Osteitis Score (GOS) (Georgalas C, Videler W, Freling N, Clin Otolaryngol 35:455-461, 2010) can be determined for the bony walls of the paranasal sinuses as previously described. As demonstrated herein, these osteitis scores correlate with Cystatin expression level in patients having chronic sinusitis, and thus a high osteitis score can be used as a proxy for high Cystatin expression levels. When an osteitis score is determined to be above a threshold (i.e., a reference level), a therapy as described herein comprising administration of a Cystatin inhibitor can be selected to treat rhinosinusitis in the subject.

One of skill in the art would readily be able to determine and select a suitable reference level. For example, a reference level can be determined as a median, average, or cutoff point for a percentile of the population (e.g., the cutoff for the top half, top tertile, top quartile, top quintile, and so on). A reference level can be selected that represents a level of Cystatin expression, eosinophilia, KOS, or GOS in a subject that would be likely to benefit from treatment with a Cystatin inhibitor, and levels at or above that reference level indicate that the subject should be treated with a method comprising administration of a Cystatin inhibitor as described herein.

Cystatin Inhibitors

A number of different cystatin inhibitors are known in the art and can be used in the present methods. For example, antibodies that bind to and inhibit cystatin activity are known in the art, including those listed below:

Recombinant Cystatins

| Name | Vendor | Cat. No |
|---|---|---|
| Recombinant human Cystatin SA protein | Abcam | ab180061 |
| (ab180061) | | |
| CST1 recombinant protein :: Cystatin SN/CST1 | Mybiosource | MBS2545742 |
| Recombinant Protein | | |
| Recombinant Human Cystatin SN Protein, CF | R&D Systems | 1285-PI-010 |
| Human Cystatin SN/CST1 Protein (His Tag) | Sinobiological | 11568-H08H-20 |
| Novus Biologicals ™ Cystatin SN Protein | Novus | NBP17883110 |
| Recombinant Human Cystatin SN protein | Abcam | ab151875 |
| (ab151875) | | |

Monoclonal Antibodies

| Name | Vendor | Cat. No |
|---|---|---|
| Human Cystatin SA Antibody | R&D Systems | MAB1201 |
| Human Cystatin SN Antibody | R&D Systems | MAB1285 |
| anti-CST1 antibody :: Mouse anti-Human Cystatin | Mybiosource | MBS690498 |
| SN Monoclonal Antibody | | |
| anti-CST2 antibody :: Mouse anti-Human Cystatin | Mybiosource | MBS2025776 |
| 2 Monoclonal Antibody | | |
| Cystatin SN Antibody (213139) | Novus | 213139 |
| Cystatin SA Antibody (OTI1D6) | Novus | OTI1D6 |
| Monoclonal Anti-CST2 antibody produced in mouse | Sigma | SAB4100393 |
| Anti-Cystatin SA antibody (MM0233- 5M60) | Abcam | ab89302 |
| (ab89302) | | |

Polyclonal Antibodies

| Name | Vendor | Cat. No |
|---|---|---|
| CST1 Polyclonal Antibody | Invitrogen | PA5-47113 |
| Rabbit Anti-Human CST1/Cystatin SN (Internal) | RayBiotech | 119-15568 |
| Anti-CST1/Cystatin Sn Antibody | BoosterBio | A05560 |
| Anti-CST1 antibody produced in rabbit | Sigma | SAB4500520 |
| Anti-CST1 antibody produced in mouse | Sigma | SAB1405670 |
| CST1/Cystatin SN Antibody (aa31-80) IHC-plus ™ | LifeSpan | LS-B5996 |
| LS-B5996 | BioSciences | |
| Anti-Cystatin SN antibody (ab124281) | Abcam | ab124281 |
| CST1 Antibody | CusaBio | CSB-PA007380 |
| Cystatin SN Antibody (Unconjugated) | Novus | AF1285 |
| Anti-CST2 antibody produced in rabbit | Sigma | HPA043706 |
| Anti-CST2/Cystatin Sa Antibody | BoosterBio | A08736 |
| Anti-Cystatin SA Antibody | Sinobiological | 11567-RP02 |
| Cystatin SA/CST2 Antibody LS-C334802 | LifeSpan | LS-C334802 |
| | BioSciences | |
| CST2 Antibody | CusaBio | CSB-PA006090ESR2HU |
| Polyclonal Antibody to Cystatin 2 (CST2) | USCN | PAJ323Hu01 |
| Anti-Cystatin SA antibody (ab206845) | Abcam | ab206845 |

To determine whether an inhibitor such as an antibody has cystatin inhibitory activity, a protease assay can be used; suitable assays are known in the art and include commercially available assays such as the EMD Millipore Calbiochem Protease Assay, which detects a quantitative array of a wide variety of proteases in biological samples, utilizing FTC-casein as a substrate. Enzyme activities that have been detected with this assay kit include: chymotrypsin, elastase, plasminogen, PRONASE® Protease, subtilisin, thermolysin, and trypsin.

Inhibitory nucleic acids, such as siRNA, shRNA, antisense oligonucleotides, morpholino oligonucleotides, and oligonucleotides with one or more modifications, e.g., modified bases or backbones, e.g., LNA, PNA, gapmers, and mixmers, can also be used.

Subjects to be Treated Using Cystatin Inhibitors

In some embodiments, a subject having rhinosinusitis, e.g., CRSwNP, is identified and treated by administration to the subject of an effective amount of a Cystatin inhibitor. The subject having rhinosinusitis, e.g., CRSwNP, can be identified by one of skill in the art based on known methods, e.g., based on detection of the presence of symptoms, by endoscopy, or by computed tomography, or using a method described herein, e.g., detection of CST1/2, PRDX5 (Peroxiredoxin-5); and/or GP6 (Platelet glycoprotein VI), e.g., in mucus derived exosomes, (whole) nasal mucus, or nasal biopsy tissue. See, e.g., McClay et al., Nasal Polyps, emedicine.medscape.com/article/994274-overview (Dec. 14, 2017); Newton and Ah-See, Ther Clin Risk Manag. 2008 April; 4(2): 507-512. The efficacy of the treatment may be monitored by methods known in the art, e.g., by monitoring symptoms, by endoscopy or computed tomography. Improvements of the subject include a better symptom score, e.g., a better SNOT-22 or VAS score; a reduction in inflammation or nasal polyp burden as revealed by endoscopy, e.g. a better Lund-Kennedy score; or a reduction in mucosal thickening or sinus opacification as revealed by computed tomography (CT), e.g. a better Lund-Mackay score. The 22-item Sinonasal Outcomes Test (SNOT-22) is a questionnaire encompassing 22 major symptoms on rhinosinusitis and nasal polyps, and serves as a valuable tool to measure the severity of a subject's symptoms and their impact on health-related quality of life (Quintanilla-Dieck, et al., International Forum of Allergy & Rhinology 2012; 2(6):437-443). The SNOT-22 assessed 12 nasal- and sinus-related symptoms (nasal blockage, loss of sense of taste and smell; need to blow nose, sneezing, runny nose, cough, postnasal discharge, thick nasal discharge, ear fullness, dizziness, ear pain, and facial pain/pressure) and 10 psychological and behavioral symptoms (difficulty falling asleep, waking up at night, lack of a good night's sleep, waking up tired, fatigue, reduced productivity, reduced concentration, frustrated/restless/irritable, sad, and embarrassed) with participants scoring each symptom on a scale of 0 (absent) to 5 (severe) on average for the last week, for a total score range of 0 to 100. The SNOT-22 score is the mean for the 22 scores (Piccirillo et al., Otolaryngol Head Neck Surg 2002; 126:41-47). The 10-symptom visual analog (VAS) scale is a questionnaire based on the major and minor symptom diagnostic criteria for CRS as described by the American Academy of Otolaryngology—Head and Neck Surgery TFR. The VAS assessed subject-reported severity of each of the following symptoms on average experienced during the prior week: nasal drainage of pus, nasal obstruction/congestion, impaired sense of smell, facial pressure/pain, headache, bad breath, weakness/fatigue, dental pain, ear fullness/pain, and cough (Ryan, et al., Laryngoscope 2011; 121:674-678). The Lund-Kennedy endoscopy scoring system quantifies the pathologic states of the nose and paranasal sinuses as assessed by nasal endoscopy, focusing on the presence of polyps, discharge, edema, scarring or adhesions, and crusting (Ryan, et al., 2011). The Lund Mackay CT scoring system is the most widely used CT grading system for chronic rhinosinusitis. This scoring system consists of a scale of 0-2 dependent on the absence (0), partial (1) or complete (2) opacification of the sinus system and the osteomeatal complex as assessed by CT imaging (Hopkins et al., Otolaryngology—Head and Neck Surgery 2007; 137:555-561). In preferred embodiments, subjects treated using a method or composition described herein have polyps.

Samples and Isolation of Nasal Mucus Exosomes

In some embodiments, a subject is diagnosed and/or treated based on the presence of cystatin, e.g., CST1 and/or CST2, in nasal mucus, nasal biopsy, or nasal mucus derived exosomes (NMDEs). As used herein the term “sample”, when referring to the material to be tested for the presence of a biological marker using the method of the invention, can mean a sample comprising (whole) nasal mucus, nasal biopsy, or NMDEs. The NMDEs can be easily sampled and isolated using a sponge or other absorbent device capable of absorbing mucus placed in the nasal cavity. An exemplary sponge is a compressed sterile 2×2×5 mm or 2×3×15 mm poly-vinyl-alcohol sponge (e.g., commercially available from Medtronics, and designed to be used in the nose for hemostasis and stenting after sinus surgery or in the setting of nose bleeds). The sponge or lavage can be stored, e.g., at −80° C., preferably in the presence of a biomarker preservative, e.g., a protease inhibitor or nuclease inhibitor (such as RNase inactivating enzymes) until isolation.

A number of methods can be used to isolate NMDEs from the sample, e.g., centrifugation (e.g., traditional ultracentrifugation (UCF) as described in (25)); chromatography; filtration; polymer-based precipitation; and immunological separation methods; see Yakimchik, Exosomes: isolation and characterization methods and specific markers, 2016-11-30, dx.doi.org/10.13070/mm.en.5.1450, and references cited therein. An exemplary polymer based exosome precipitation system is the ExoQuick from System Biosciences. In an exemplary UCF method, mucus and irrigant samples can be diluted, e.g., in 150 μL of 1× phosphate buffered saline (PBS) with Protease Inhibitor Cocktail. Cellular debris can be pelleted by centrifugation, e.g., at 45 min at 12,000×g at 4° C. The supernatant can then be suspended in PBS, e.g., 4.5 mL of PBS in polypropylene tubes, and ultracentrifuged, e.g., for 2 hours at 110,000×g, at 4° C. The supernatant can then be collected and the pellet resuspended in PBS, e.g., in 4.5 mL 1×PBS. The suspension can be filtered, e.g., through a 0.22-μm filter, and collected in a fresh tube. The filtered suspension can then be centrifuged again, e.g., for 70 min at 110,000×g at 4° C. The supernatant can then be collected and the pellet resuspended in a buffer, e.g., in PBS, e.g., in 200 μl PBS with protease inhibitor.

Various methods known within the art can be used for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample can be first isolated according to standard methods, for example using lytic enzymes, chemical solutions, or isolated by nucleic acid-binding resins following the manufacturer's instructions. Exemplary sequences of CST1 and CST2 and other biomarkers are provided below; see also FIG. 10. Other isoforms of these proteins as known in the art can also be used.

### Exemplary Sequences of Biomarkers

| | NCBI Reference | NCBI Reference |
|---|---|---|
| Gene | Sequence mRNA | Sequence protein |
| CST1 cystatin-SN precursor | NM_001898.2 | NP_001889.2 |
| CST2 cystatin SA precursor | NM_001322.2 | NP_001313.1 |
| PRDX5 (Peroxiredoxin-5) | NM_012094.5 | NP_036226.2 |
| GP6 (Platelet glycoprotein VI) | NM_001083899.2 | NP_001077368.2 |

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art. Next generation ultrasensitive ELISA can also be used, e.g., a single molecule array (SIMOA) digital ELISA may also be used. For example, for each assay, capture antibody is first covalently conjugated to magnetic particles utilizing a standard EDC coupling procedure and detection antibody was biotinylated. In the first step of the assay, antibody coated paramagnetic capture beads, biotinylated detection antibodies, and samples are combined, during which target molecules present in the sample are captured by the capture beads and labeled with the biotinylated detection antibodies. After washing, a conjugate of streptavidin-β-galactosidase (SβG) is mixed with the capture beads where SβG bound to the biotin, resulting in enzyme labeling of captured target molecules. Following a second wash, the capture beads are resuspended in a resorufin β-D-galactopyranoside (RGP) substrate solution and transferred to the Simoa array disc for detection.

In some embodiments, an IHC method may be used. IHC provides a method of detecting a biological marker in situ. The presence and exact cellular location of the biological marker can be detected. Typically, a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047).

In some embodiments, Slow Off-rate Modified Aptamer (SOMAmer)-based capture array called ‘SOMAscan’ (SomaLogic, Inc, Boulder, Colo.) can be used. This approach uses chemically modified nucleotides to transform a protein signal to a nucleotide signal that can be quantified using relative florescence on microarrays. This assay has been shown to have a median intra- and inter-run coefficient of variation of ˜5%.

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of a biomarker. Measurement of the level of a biomarker can be direct or indirect. For example, the abundance levels of biomarkers can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes are most commonly used.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

Comparison to a Reference Level

In some embodiments, the presence and/or level of a biomarker is comparable to the presence and/or level of the protein(s) in the disease reference, and the subject has one or more symptoms associated with CRSwNP, then the subject has CRSwNP.

In some embodiments, the subject has no overt signs or symptoms of CRSwNP, but the presence and/or level of one or more of the proteins evaluated is comparable to the presence and/or level of the protein(s) in the disease reference, then the subject has an increased risk of developing CRSwNP or has an early stage of the disease, e.g., CRS. In some embodiments, once it has been determined that a person has CRSwNP, or has an increased risk of developing CRSwNP, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of a biomarker, e.g., a control reference level that represents a normal level of the biomarker, e.g., a level in an unaffected subject or a subject who is not at risk of developing a disease described herein, and/or a disease reference that represents a level of the proteins associated with conditions associated with CRS, e.g., with CRSwNP.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have CRS, e.g., does not have CRSwNP.

A disease reference subject is one who has (or has an increased risk of developing) CRSwNP. An increased risk is defined as a risk above the risk of subjects in the general population.

Thus, in some cases the level of a biomarker in a subject being less than or equal to a reference level of the biomarker is indicative of a clinical status (e.g., indicative of a disorder as described herein, e.g., CRSwNP. In other cases, the level of the biomarker in a subject being greater than or equal to the reference level of the biomarker is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of the biomarker in a subject being equal to the reference level of the biomarker, the “being equal” refers to being approximately equal (e.g., not statistically different).

The predetermined value can depend upon the particular population of subjects (e.g., human subjects) selected. For example, an apparently healthy population will have a different ‘normal’ range of levels of the biomarker than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Conventional Treatment of Rhinosinusitis

The present methods can be used in combination with present conventional treatments of rhinosinusitis, e.g., as follows. Most subjects with inflammatory rhinosinusitis caused are treated with a nasal decongestant. However, nasal decongestants may dry out the affected areas and damage tissues. With prolonged use, nasal decongestants become ineffective, and the tendency is to increase the frequency of use. Withdrawal from over-frequent decongestant use can itself cause symptoms of rhinosinusitis and the return of nasal congestion, a phenomenon known as the “rebound effect.” Short-acting nasal decongestants may cause rebound effect after only eight hours. Eventually, the inflammation can become worse than before the decongestant was taken. Thus, nasal decongestants are generally recommended for no more than one to three days of use because of this risk.

Steroid nasal sprays are commonly used to treat inflammation in chronic sinusitis. For subjects with severe chronic sinusitis, doctors may prescribe oral steroids, such as prednisone. Since oral steroids have serious side effects, they are prescribed only when other medications have not been effective.

When medications fail, surgery may be the only alternative in treating chronic sinusitis. Presently, the most commonly done surgery is functional endoscopic sinus surgery, in which the sinuses are reached through the nasal passage via endoscopy, and the diseased and thickened tissues and polyps from the sinuses are removed to enlarge the sinus passageway to the nostril and allow for drainage and improved topical drug delivery. This type of surgery is less invasive than traditional open sinus surgery techniques. Symptoms of chronic sinusitis sometimes persist after surgery, however, because of continued inflammation, growth of new nasal polyps, or scarring from the procedure.

Methods for Selecting a Subject for Participation, or Stratifying Subjects, in a Clinical Study

Also provided are methods of selecting a subject for participation in, or stratifying subjects in, a clinical study of a treatment for rhinosinusitis or CRSwNP. Such methods can include determining a level of epithelial Cystatin expression in a sinus mucosal biopsy sample from a subject, comparing the Cystatin level in the sample to a reference Cystatin level, and selecting for participation a subject having an elevated Cystatin level in the sample compared to the reference Cystatin level in a clinical trial of a treatment for rhinosinusitis or CRSwNP, or stratifying subjects in a clinical trial based on Cystatin levels. In some embodiments, a subject can be excluded from participation in a clinical study of a treatment for rhinosinusitis or CRSwNP if the subject has no significant change or a decrease in the Cystatin level in the sample compared to the reference Cystatin level.

Also provided are methods of selecting a subject for participation in, or stratifying subjects in, a clinical study for a treatment for rhinosinusitis or CRSwNP. Such methods include determining a Cystatin level in a first sample, e.g., a biopsy, mucus, or mucosal exosome sample, obtained from a subject at a first time point, determining a Cystatin level in a second sample obtained from the subject at a second time point, comparing the Cystatin level in the first sample to the Cystatin level in the second sample, and selecting a subject having an elevated Cystatin level in the second sample compared to the Cystatin level in the first sample for participation in a clinical trial of a treatment for rhinosinusitis or CRSwNP, or stratifying subjects in a clinical trial based on changing Cystatin levels. In some embodiments, a non-invasive exosomal sample is preferred as this can be done serially, prospectively, and non-invasively. In some embodiments, a subject can be excluded from participation in a clinical study of a treatment for rhinosinusitis or CRSwNP if the subject has no significant change or a decrease in the Cystatin level determined at the second time point compared to the Cystatin level determined at the first time point. In some embodiments, the treatment for rhinosinusitis or CRSwNP is a pharmacological treatment (e.g., administration of one or more pharmaceutical agents) or the implantation of an eluting implant, stent, or spacer.

In some embodiments, additional clinical scores can be used to assist in selecting a subject for participation in, or stratifying subjects in, a clinical study of a treatment for rhinosinusitis or CRSwNP. For example, sinus mucosal biopsy samples can be processed for hematoxylin and eosin staining and the average number of eosinophils per high powered field can be calculated and used in selecting a subject for participation in, or stratifying subjects in a clinical study of a treatment for rhinosinusitis or CRSwNP. For example, subjects with levels of eosinophilia above a reference level can indicate that the subject should be selected or stratified.

In some embodiments, computed tomography (CT) can be performed to score osteitis in a subject having rhinosinusitis or CRSwNP. The osteitis score, e.g., Kennedy Osteitis Score (KOS) or Global Osteitis Score (GOS) can be used in selecting a subject for participation in, or stratifying subjects in, a clinical study of a treatment for rhinosinusitis or CRSwNP. For example, subjects with GOS or KOS above a reference level can indicate that the subject should be selected or stratified.

The clinical studies may be performed by a health care professional (e.g., a physician, a physician's assistant, a nurse, a phlebotomist, or a laboratory technician) in a health care facility (e.g., a hospital, a clinic, or a research center). The biopsy samples may be obtained from subjects that present with one or more (e.g., at least two, three, four, or five) symptoms of rhinosinusitis or CRSwNP.

Pharmaceutical Compositions, Dosage, and Methods of Administration

The methods of treatment described herein also include the use of pharmaceutical compositions, which include Cystatin inhibitors described herein as active ingredients. In some embodiments the composition also includes one or more supplementary active compounds incorporated therein, e.g., one or more corticosteroids and/or one or more antibiotics. The corticosteroid can be, e.g., selected from dexamethasone, prednisone, prednisolone, triamcinolone, cortisol, budesonide, mometasone, fluticasone, flunisolide, or betamethasone. The antibiotic can be, e.g., selected from erythromycin, doxycycline, tetracycline, penicillin, beta-lactam, macrolide, fluoroquinolone, cephalosporin, and sulfonamide. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders, for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems.

The pharmaceutical compositions can be included in a container, pack, or dispenser together with instructions for administration.

In some embodiments, a kit for treating rhinosinusitis in a subject is provided. Such a kit comprises a pharmaceutical composition comprising an effective amount of a Cystatin inhibitor, optionally a corticosteroid and/or an antibiotic, and a device for delivering the pharmaceutical composition to the subject's nasal passage and sinuses, such as a nebulizer, an inhaler, or an OptiNose. The device may deliver the pharmaceutical composition to the subject's nasal passage and sinuses in a liquid or an aerosolized form.

In non-limiting examples, the pharmaceutical composition containing at least one pharmaceutical agent is formulated as a liquid (e.g., a thermosetting liquid), as a component of a solid (e.g., a powder or a biodegradable biocompatible polymer (e.g., a cationic biodegradable biocompatible polymer)), or as a component of a gel (e.g., a biodegradable biocompatible polymer). In some embodiments, the at least composition containing at least one pharmaceutical agent is formulated as a gel selected from the group of an alginate gel (e.g., sodium alginate), a cellulose-based gel (e.g., carboxymethyl cellulose or carboxyethyl cellulose), or a chitosan-based gel (e.g., chitosan glycerophosphate). Additional, non-limiting examples of drug-eluting polymers that can be used to formulate any of the pharmaceutical compositions described herein include, carrageenan, carboxymethylcellulose, hydroxypropylcellulose, dextran in combination with polyvinyl alcohol, dextran in combination with polyacrylic acid, polygalacturonic acid, galacturonic polysaccharide, polysalactic acid, polyglycolic acid, tamarind gum, xanthum gum, cellulose gum, guar gum (carboxymethyl guar), pectin, polyacrylic acid, polymethacrylic acid, N-isopropylpolyacrylomide, polyoxyethylene, polyoxypropylene, pluronic acid, polylactic acid, cyclodextrin, cycloamylose, resilin, polybutadiene, N-(2-Hydroxypropyl)methacrylamide (HPMA) copolymer, maleic anhydrate-alkyl vinyl ether, polydepsipeptide, polyhydroxybutyrate-polycaprolactone, polydioxanone, polyethylene glycol, polyorganophosphazene, polyortho ester, polyvinylpyrrolidone, polylactic-co-glycolic acid (PLGA), polyanhydrides, polysilamine, poly N-vinyl caprolactam, and gellan.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

Methods

The following methods were used in the examples set forth herein.

Patient Tissue and Mucus Sampling

Tissue and mucus sampling was approved by the Massachusetts Eye and Ear Infirmary Institutional Review Board. Matched tissue and mucus exosomal samples were taken from patients undergoing sinonasal surgery and had not been exposed to antibiotics or any topical/systemic steroids for at least 4 weeks. Inclusion criteria included patients diagnosed with CRSwNP by the International Consensus Statement on Allergy and Rhinology (ICAR:RS) (35) criteria and healthy patients (i.e. Controls) undergoing surgery for non-inflammatory disease. Exclusion criteria included ciliary dysfunction, autoimmune disease, cystic fibrosis, or immunodeficiency. Among controls, additional exclusion criteria included the presence of allergy or asthma.

Tissue, Mucus, and Exosome Collection Technique

Mucus samples were taken prior to antibiotic or steroid administration by applying a compressed polyvinyl alcohol sponge (PVA, Medtronic, Minneapolis, Minn.) to either the posterior nasal cavity (POST) adjacent to the middle turbinate or anterior (ANT) internal valve taking care not to abrade the mucosa or contaminate the sponge with blood. Adjacent nasal polyp (in CRSwNP) and nasal mucosa (in controls) tissue samples were then harvested using a blakesly forceps while minimizing crush injury to the tissue.

Alternatively, mucus samples were taken prior to tissue sampling by placing compressed PVA sponges against the middle meatus for 5 minutes taking care not to abrade the mucosa or contaminate the sponge with blood.

Exosome Purification Technique from Whole Mucus

The exosome purification procedure was adapted from the ultracentrifugation (UCF) procedure described by Théry et al (36) and previously reported by our group (37). Mucus samples were extracted from the PVA sponges by centrifugation (1500 g at 4° C. for 30 minutes). The mucus was then diluted in 150 μL of 1×phosphate buffered saline (PBS, Life Technologies, Carlsbad, Calif.) with Protease Inhibitor Cocktail (1:100, Sigma, St. Louis, Mo.). Cellular debris was pelleted by centrifugation at 45 min at 12,000×g at 4° C. The supernatant was then suspended in 4.5 mL of PBS in polypropylene tubes (Thinwall, 5.0 mL, 13×51 mm, Beckman Coulter, Indianapolis, Ind.) and ultracentrifuged for 2 hours at 110,000×g, at 4° C. The supernatant was collected and the pellet was resuspended in 4.5 mL 1×PBS. The suspension was filtered through a 0.22-μm filter (Fisher Scientific, Pittsburgh, Pa.) and collected in a fresh ultracentrifuge tube. The filtered suspension was then centrifuged for 70 min at 110,000×g at 4° C. The supernatant was collected and the pellet was resuspended in 175 μl PBS M-per with protease inhibitor for further proteomic analysis.

Western Blots

Western blots were performed in an independent group of CRSwNP and control (n=6 per group) tissue and mucus derived exosomal samples in order to validate the proteomic results. Homogenization (T25 Basic, IKA Labortechnik, Staufen, Germany) of 30-40 mg of each tissue sample was accomplished in 1.0 mL lysis buffer (T-PER™ Mammalian Protein Extraction Reagent (Thermo Scientific, Bonn, Germany) and protease inhibitor cocktail Complete1 (Roche, Mannheim, Germany)). They were then incubated at 4° C. for 2 hours and centrifuged at 16000 g for 1 hour. The total protein concentration of the supernatant was determined with bicinchoninic acid assays (Thermo Fisher Scientific, Bonn, Germany). 50 ug of lysed tissue protein were used for each patient. Exosomes were isolated as described above, and 2 ug of lysed exosomal protein was used for each patient. After denaturation at 90° C. with SDS loading buffer including mercaptoethanol for 5 minutes, lysates were applied on 8-15% SDS-Page and transferred to nitrocellulose membranes (Protran-BA-83, Schleicher & Schuell). GAPDH (monoclonal rabbit antibody against GAPDH, clone D16H11, New England Biolabs GmbH Frankfurt, Germany) staining served as control. The primary detection antibodies were mouse Anti-CST1, clone 213139 (R&D Systems, Abingdon, UK) for Cystatin-SN (CST 1) and Peroxiredoxin-5 (mouse Anti-PRDX5, clone B-7, Santa Cruz Biotechnology, Heidelberg, Germany); respectively followed by the secondary antibody (Peroxidase-labeled anti-mouse/rabbit IgG, F(ab′)2 antibody (KPL, Gaithersburg, USA). The blot was incubated with SuperSignal West Dura Extended Duration Substrate (Thermo Fisher Scientific, Bonn, Germany) and the signals were imaged using ChemStudio PLUS (Analytikjena, Jena, Germany). Quantification of band intensity was performed using VisionWorks 8.2 (Analytikjena, Jena, Germany).

Immunohistochemistry

Paraffin embedded formalin fixed tissue samples were sectioned, deparaffinized, and rehydrated prior to staining. Antigen retrieval was performed using Proteinase K (Thermo Fisher Scientific, Waltham, Mass.) for 5 minutes at room temperature. After washing twice, the tissue was blocked with BLOXALL Endogenous Peroxidase and Alkaline Phosphatase Blocking Solution (Vector Laboratories, Burlingame, Calif.) for 10 minutes at room temperature in a humidified chamber. The slides were washed once and then blocked with Horse Serum (2.5% Normal Horse Serum Blocking Solution, Vector Laboratories) for 20 minutes at room temperature in a humidified chamber. The blocking solution was removed and the primary antibody (1:7500 Anti-Cystatin SA antibody or 1:3000 Anti-Cystatin SN antibody Abcam, Cambridge, Mass.) in 1% BSA/PBS was incubated on the slides overnight at 4° C. The secondary antibody, ImmPRES HRP Anti-Mouse IgG (Peroxidase) Polymer Detection Kit (Vector Laboratories) was applied for 30 minutes at room temperature. After rinsing 3 times. The slides were developed with ImmPACT DAB Peroxidase (HRP) Substrate (Vector Laboratories) for 1 minute at room temperature and stop with ddH2O for 5 minutes. The slides were counter stained with hematoxylin, dehydrated with xylenes, and coverslipped with permount.

Proteomic Analysis

SOMAscan proteomic analysis (SomaLogic; Boulder, Colo.) on nasal tissue and isolated exosomes was run using the SOMAscan Assay Cells & Tissue Kit, 1.3k (SomaLogic #900-00009) following the recommended protocol from the manufacturer. For nasal tissue extracts and mucus, 2.4 ug of protein from each sample was run. With exosomes isolated from nasal mucous, 1.7 ug per sample of protein was used in the SOMAscan assay. Three provided kit controls and one no-protein buffer control were run in parallel with the samples per plate. Median normalization and calibration of the SOMAscan data was performed according to the standard quality control (QC) protocols at SomaLogic.

DNA and RNA Extraction

DNA and RNA were extracted simultaneously from a single fresh frozen tissue sample using Qiagen's AllPrep DNA/RNA/miRNA Universal Kit. Since there is no need to divide the sample into two for separate purification procedures, maximum yields of DNA and RNA can be achieved. Tissues are first homogenized and then lysed in a highly denaturing buffer, which destroys DNases and RNases present in the sample to allow for simultaneous purification. The lysate is passed through the DNA binding column, leaving the DNA behind and the RNA in the flow-through. Ethanol is added to adjust binding conditions and the flow-through sample is passed through the RNA binding column. Wash steps eliminate contaminants present on both columns and the DNA and RNA are eluted from their respective columns.

Transcriptome Analysis

RNA samples were prepared from matched tissue samples using the Illumina TruSeq Stranded mRNA sample preparation kit (Illumina, San Diego, Calif.) to obtain 150 ng of purified total RNA. ThermoFisher External RNA Controls Consortium (ERCC) controls (Pittsburgh, Pa.) were added prior to Poly(A) selection providing additional control for variability. RNA quality and insert-size were assessed by Caliper LabchipGXII (Perkin-Elmer, Waltham, Mass.) producing an RNA quality score (RQS) value (equivalent to RNA integrity number). RNA quantity was determined by RiboGreen. Samples were then sequenced using the Illumina HiSeq4000. Trimmed reads were aligned to the human reference genome (hg19) using HISAT27 (version 2.1.0). The output SAM files were converted to BAM files and sorted using SAMtools.

Variant Calling for Exome Data

The trimmed reads from the exome data were aligned to the human reference genome hg19 using BWA (38) (mem -M -t 4), Version 0.7.1. GATK (version 3.5) was invoked to perform variant calling. Picard Tools (39) (version 2.3.0) were used to process the bam alignment files to mark PCR duplicates. Broad Institute's Best Practices guidelines recommend that “hard filters” be applied with GATK VariantFiltration. The following “hard filters” were applied in the current run. For SNVs, variants meeting the following criteria were filtered out: Fisher Strand value (FS)>60.0, Qual By Depth value (QD)<2.0, Mapping quality value (MQ)<40, value of Rank Sum Test for Mapping qualities (MQRankSum)<−12.5, or value of Rank Sum Test for site position within reads (ReadPosRankSum)<−8.0. For InDels, variants meeting the following criteria were filtered out: FS >200.0, QD<2.0, or ReadPosRankSum <−20.0.

Variant Prioritization

We used ANNOVAR (40) to annotate the functional alteration for the variants. At the completion of ANNOVAR analysis, the results were summarized, and retained variants were subsequently evaluated using a series of filters implemented using scripts developed by our group based on previously described techniques (41,42). The first of these filters examines potential functional consequences of the variants. The variants are classified based on the location of their occurrences, into “exonic”, “intronic”, “5′ UTR”, “3′ UTR”, “splicing site”, “intergenic”, “upstream” or “downstream” mutations, where “upstream” and “downstream” mutations refer to mutation occurring within a 1-kb distance from a transcription start site and a transcript end site, respectively. Exonic mutations were further categorized into 8 subcategories: frameshift deletions, frameshift insertions, nonframeshift deletions, nonframeshift insertions, nonsynonymous SNVs, synonymous SNVs, stopgain and stoploss mutations. In the filter we implemented, we retained variants (SNVs and InDels) of high or moderate impacts, i.e. damaged nonsynonymous SNVs, stopgain, stoploss, damaged splice variants, frameshift insertions and deletions. The damaged variants were defined as the variants either called “deleterious” by SIFT (43) or called “possibly damaging” or “probably damaging” by PolyPhen2 HDIV (44). The SIFT and PolyPhen2 HDIV information was provided by ANNOVAR. The second filter requires that the minor allele frequency (MAF) of the variants is less than 0.5%, according to the European subpopulation of the 1000 Genomes Project (45) and the Non-Finnish European subpopulation in Exome Aggregation Consortium (ExAC) (46). Variants that are not documented in these two projects are also retained. The third filter we implemented is an evolutionary conservation filter: we adopted a PhastCons score cut-off of 0.90 to select variants in evolutionarily conserved regions. We retained all variants passing these filters in the any of the 10 Polyp patents but are not present in any of the 10 control subjects as candidate variants. Finally, these candidate variants were categorized into homozygous and heterozygous variants.

CST1 and 2 Exome Analysis

Both CST1 and CST2 are minus chain transcript genes located in chromosome 20. Each gene has one transcript from the human UCSC refGene (hg19). We defined the promoter region for each gene as 2,000 base pairs (bp) upstream of the transcript start site (TSS). The TSSs for CST1 and CST2 transcripts are 23731503 and 23807297, respectively. The transcript end sites (TTSs) for CST1 and CST2 transcripts are 23728452 and 23804656, respectively. The two transcripts are 3052 and 2642 bp long, comparable to the 2000 bp arbitrarily chosen as the promoter length.

Protease Assay

Papain (Papain from papaya latex, Sigma, St. Louis, Mo.) was used as a model protease to determine protease activity using a commercially available kit (Protease Assay Kit, EMD Millapore, Burlington, Mass.). The kit was run according to the manufacturer's instructions, with the addition of a Papain standard curve. After determining the optimal dose of Papain (0.625 mg/mL), recombinant CST1 (R&D Systems, Minneapolis, Minn., Mybiosource, San Diego, Calif., or Sinobiological, Wayne, Pa.) or CST2 (Abcam, Cambridge, Mass.) was added to the determine if the Papain protease activity could be inhibited. The recombinant cystatins were incubated with Papain at 37° C. for 30 minutes. The optimal dose for recombinant CST1 was determined to be 12.5 μg/mL and 25 μg/mL for CST2. To determine if the protease inhibition could be abrogated, monoclonal antibodies for CST1 (R&D Systems and Mybiosource) or CST2 (R&D Systems and Mybiosource) were incubated with the recombinant proteins at 37° C. for 30 minutes, and then Papain was added to the mixture and incubated at 37° C. for 30 minutes.

Functional Inflammatory Assays

The research protocol for this study was approved by the Massachusetts Eye and Ear Infirmary Institutional Review Board. Control and CRSwNP ethmoid mucosal explant specimens were obtained from patients undergoing functional endoscopic sinus surgery. For each patient, 14 organotypic explants were prepared using a 5-mm biopsy punch, taking care to ensure that each contained an intact epithelial layer. The explants were exposed to the vehicle control (serum- and hydrocortisone-free bronchial epithelial growth medium; Lonza, Basel, Switzerland) for 1 hour at 37° C. and the media supernatant was then collected. The explants were then exposed to one of the CST1 (R&D Systems, Mybiosource, or Sinobiological) at 12.5 or 6.25 μg/mL or CST2 (Abcam) at 25 or 12.5 μg/mL recombinant proteins for 24 hours at 37° C. and the media supernatant was then collected. The supernatants were then exposed to IL-5, 6, 8, and TSLP ELISAs (eBioscience, San Diego, Calif.) and Micro BCA Protein Assay Kit (Pierce, Rockford, Ill.) to quantify cytokine expression after exposure to recombinant CST1 or 2. Hour 24 was normalized by hour 1 to determine the Th2 expression. All samples were analyzed in duplicate and statistical significance was determined using the Student's t-Test when compared to the vehicle control.

Statistical Analysis

Protein expression profiling was done using SomaLogic's SOMAscan™ platform (47) for 1,317 proteins including 12 controls. Protein abundance was measured in normal (controls, labeled C) and polyp (labeled P) samples. Data was obtained in two sets: Set1 involved 10 C and 10 P matched tissue, whole mucus (all POST), and exosome samples. Set2 involved 10 C and 10 P exosome samples with 4 C and 5 P samples collected from anterior nasal cavity (ANT) and the rest collected from the posterior nasal cavity (POST). One C and one P sample from Set1 were included in Set2 to assess reproducibility. Raw data (abundance signal values) were normalized using the rank invariant set method (48). Briefly, proteins that do not exhibit large differences in their signal rank between two samples were labeled as the rank invariant set and a lowess smoothing line was fitted through their expression values to define the normalization equation.

Differential protein expression was calculated using Student's t-test followed by the Benjamini Hochberg procedure (49) for multiple hypotheses testing correction. A false discovery rate (FDR) value of <0.05 was considered statistically significant. Clustering of samples and/or proteins was done using the Unweighted Pair Group Method with Arithmetic-mean (UPGMA) method with Pearson's correlation as the distance measure (Sneath P, Sokal R. Numerical Taxonomy: The Principles and Practice of Numerical Classification. WH Free Company, San Fr. 1973; 573). The expression data matrix was row-normalized prior to the application of average linkage clustering. Discriminatory power of the protein expression was also established using principal components analysis (PCA). PCA structures and organizes large datasets by converting a set of observations of possibly correlated variables into a set of values of linearly uncorrelated variables called principal components. In a multivariate, high-dimensional dataset PCA displays the data in a low-dimensional picture (2D PCA). PCA best explains the variance of a cluster and separates it against other clusters (Pearson K. LIII. On lines and planes of closest fit to systems of points in space. Philos Mag Ser 6. 1901; 2:559-72). Classification was done using Support Vector Machines (SVM)(Hsu C-W, Lin C-J. A comparison of methods for multiclass support vector machines. IEEE Trans neural networks. 2002; 13:415-25) on the PCA results. During the SVM application, different kernels (e.g., Linear, Gaussian, or Polynomial) were tested and the kernel with the best prediction accuracy was reported. K-fold cross validation and area under the curve (AUC) of the receiver operating characteristics (ROC) were applied to assess the predictive performance of protein expression.

Predictive protein set development was performed using a combination of robustness in differential expression and fold change (FC: ratio of the average expression between P and C samples) analysis. Set1 was regarded as the training set and was analyzed for potential predictive proteins (Mueller S, Nocera A, Dillon S, Wu D, Libermann T, Bleier B. Highly multiplexed proteomic analysis reveals significant tissue and exosomal coagulation pathway derangement in chronic rhinosinusitis with nasal polyps. Int Forum Allergy Rhinol. 2018; 8(12):1438-1444). The identified potential predictive proteins were separately applied on the following four sets as the test set: Set1, Set2, Set2 POST samples only, and Set2 ANT samples only. In order to identify potential predictive protein sets, samples in Set1 were split 10,000 times by 4-fold (i.e. ¼th of the samples in each group were left out in each split) and for each split a set of significantly differentially expressed proteins (p<0.01) were calculated. Proteins were assigned a “frequency” value based on the number of times they were included in the significantly differentially expressed list out of the 10,000 splits. 12 proteins based on their robustness in differential expression (top 10 highest frequency) and high FC (top 2, not included in the top 10 most frequent) were picked as the refined predictive set. 4-fold cross validation using each combination of the 12 proteins (i.e. 2¹²−1=4,095 protein sets) were applied on each of the four test sets. For each test set, 1,000 4-fold splits were tried for each of the 4,095 predictive protein set combinations. If the number of possible 4-fold splits for a test data was less than 1,000, all possible 4-fold splits were used. Average, median, 95% lower and upper confidence bounds, and 25th and 75^{th} percentile of accuracy, sensitivity and specificity were calculated. ROC curves and corresponding AUC values were obtained for predictive protein sets with good performance.

Patient Demographics

There were no significant differences between CRSwNP and control groups (n=20 per group, set 1 and 2 are displayed separately) with respect to age, gender, race, environmental allergies, or AERD. Concomitant asthma differed significantly between groups (set 1 p=0.01; set 2 p=0.029, respectively) (Table 1A and B). No patient was diagnosed with AFRS (acute fungal rhinosinusitis), all CRSwNP patients showed eosinophilia in histology.

TABLE 1A
Set 1

| Characteristics n(%) | CRSwNP | Control | | p-value |
|---|---|---|---|---|
| Mean age in years (±SD) | 46.8 ± 11.57 | 35.1 ± 13.35 | | 0.051 |
| Gender | | | | |
| Male | 7/10 (70) | 5/10 | (50) | 0.361 |
| Female | 3/10 (30) | 5/10 | (50) | 0.361 |
| Race | | | | |
| Caucasian | 10/10 (100) | 10/10 | (100) | 1 |
| Comorbiditiy | | | | |
| Asthma | 5/10 (50) | 0/10 | (0) | 0.01 |
| Environmental allergy | 1/10 (10) | 0/10 | (0) | 0.305 |
| AERD | 1/10 (10) | 0/10 | (0) | 0.305 |

TABLE 1B
Set 2

| Characteristics n(%) | CRSwNP | Control | | p-value |
|---|---|---|---|---|
| Mean age in years (±SD) | 37.3 ± 12.43 | 43.9 ± 15.18 | | 0.302 |
| Gender | | | | |
| Male | 7/10 (70) | 4/10 | (40) | 0.189 |
| Female | 3/10 (30) | 6/10 | (60) | 0.189 |
| Race | | | | |
| Caucasian | 10/10 (100) | 10/10 | (100) | 1 |
| Comorbiditiy | | | | |
| Asthma | 4/10 (40) | 0/10 | (0) | 0.029 |
| Environmental allergy | 1/10 (10) | 0/10 | (0) | 0.305 |
| AERD | 1/10 (10) | 0/10 | (0) | 0.305 |

Patient demographics for A) Set 1 (n=10 per group) and Set 2 (n=10 per group) AERD: Aspirin-exacerbated respirator, disease

### Example 1. Exosomes May be Reproducibly Isolated, Contain a Unique Proteomic Signature, and are More Homogenous then Whole Mucus

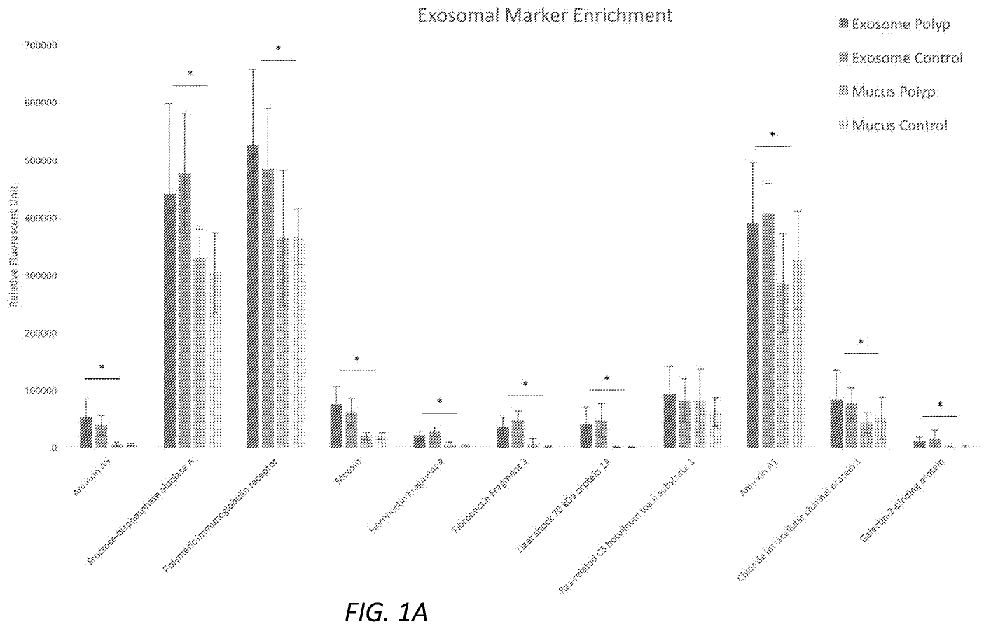

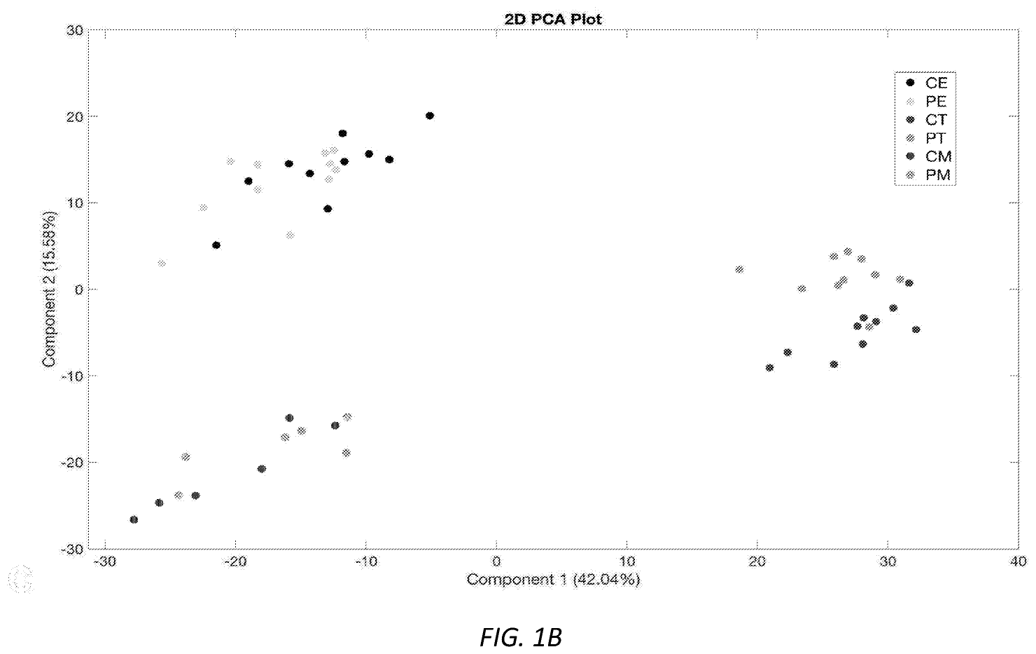

Among Set1 (training set) data, eleven established core exosomal markers' among both the CRSwNP and control patients were significantly enriched in the exosome isolate relative to the matched mucus samples (FIG. 1A) demonstrating successful purification of the exosome fraction. The overall exosomal proteome between CRSwNP and control groups correlated more strongly to one another (r=0.84, p<0.001) than with either the mucus or tissue proteome (r=0.72, p<0.001, p=0.66, p<0.001) suggesting that that the exosome isolation yielded discreet and reproducible protein expression data between patients. This was subsequently confirmed by 2 dimensional PCA. (FIG. 1B) Furthermore, the inter-patient variance among the sampled proteins was significantly lower in the exosomes than matched whole mucus samples (5.88×10⁸ vs 2.4×10⁹, p<0.05).

Among the Set1 data, seventy-five proteins were significantly up regulated in the exosomal proteome in CRSwNP (17, p<0.01; 58, p<0.05) relative to control. Similarly, forty-eight proteins were significantly down regulated (10, p<0.01; 38, p<0.05) among the polyp patients relative to control. As a result of the improved S/N evident in the exosomal proteome relative to whole mucus, 80 of these proteins overlapped with the CRSwNP tissue proteome versus only 4 in matched whole mucus. One hundred and two of these perturbed exosomal proteins have not been previously described within the CRSwNP literature.

Following invariant set normalization, the rerun samples showed high correlation between Sets 1 and 2 (0.9918 for the control and 0.9891 for the polyp sample) implying successful normalization and reproducibility. Our results showed successful separation of the polyps rendering a proteomic profile associative with the phenotype. As described in the Methods section, we identified 12 proteins with the highest FC and most robust differential expression as the refined predictive protein set. These 12 proteins, shown in Table 2, form a subset of the 50 differentially expressed proteins. All 4,095 possible combinations of the 12 proteins were used as candidate predictive protein sets.

TABLE 2
Predictive protein set with highest FC and most robust
differential expression between patient groups in Set1

| Full Name | Entrez ID | p-value |
|---|---|---|
| Caspase-3 | CASP3 | 0.0002 |
| Cystatin-SN | CST1 | 0.0075 |
| Cystatin-SA | CST2 | 0.0015 |
| Discoidin domain-containing receptor 2 | DDR2 | 0.0017 |
| Dickkopf-like protein 1 | DKKL1 | 0.0023 |
| Dual specificity tyrosine-phosphorylation- | DYRK3 | 0.0015 |
| regulated kinase 3 | | |
| Platelet glycoprotein VI | GP6 | 0.0017 |
| Pappalysin-1 | PAPPA | 0.0078 |
| cGMP-dependent 3′,5′-cyclic phosphodiesterase | PDE2A | 0.0014 |
| Peroxiredoxin-5, mitochondrial | PRDX5 | 0.0015 |
| Protein S100-A7 | S100A7 | 0.001 |
| Thrombospondin-1 | THBS1 | 0.001 |

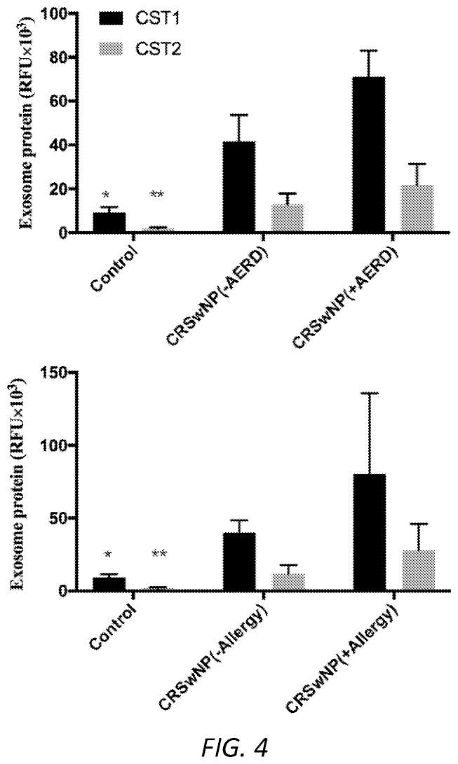

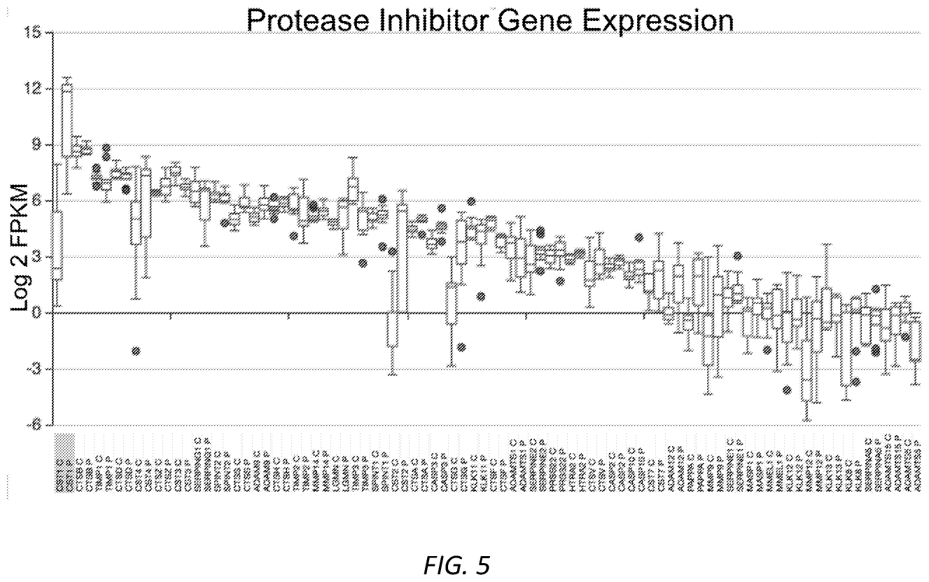

Prediction performance gradually degraded as the test data set followed the order Set1-Set2 POST Only-Set2 ALL-Set2 ANT Only. Therefore, the site of the sample (e.g. posterior versus anterior) influenced the proteomic profile, which determined the prediction performance. This is also evident from FIG. 4B, which depicts the PCA of Set2 exosomal samples only in an unsupervised fashion using all of the 1,305 proteins. This global proteomic profile clustering indicates that the site of the sample is a major factor in the overall protein expression. Indeed, a direct comparison between these groups reveal 74, 27, and 153 significantly differentially expressed proteins (p<0.01) between POST and ANT samples in control, polyp, and all samples in Set2, respectively. We also identified proteins significantly differentially expressed (p<0.01) between polyp and control samples across POST (28 proteins) and ANT samples (5 proteins) separately in Set2. The 5 proteins associated with the polyp phenotype when only ANT samples were considered were not among the 28 proteins significantly differentially expressed between POST polyp and POST control samples. These results imply that the difference in protein abundance due to nasal polyps is more profound in the POST samples and exhibit a site-dependent behavior.

We next generated ROC curves and calculated the AUC values for the best performing predictive exosomal protein set for Set1, which was composed of the proteins CST1 (Cystatin-SN), PRDX5 (Peroxiredoxin-5) and GP6 (Platelet glycoprotein VI). The average 4-fold cross validation accuracy of this protein set on Sett samples were 94.47%. In, we show the ROC curve and AUC values for the SVM prediction results for the top performing test set. This 3-protein set predictor achieves an AUC value of 99% on Sett and 90% on Set2 POST samples (FIG. 4D), while the performance quickly deteriorates for Set2 samples (82%) and Set2 ANT samples (20%). These results also confirmed the effect of location on the proteomic profile.

### Example 2. Western Blot Confirmation and Histopathologic Localization of CST 1 and 2 Expression

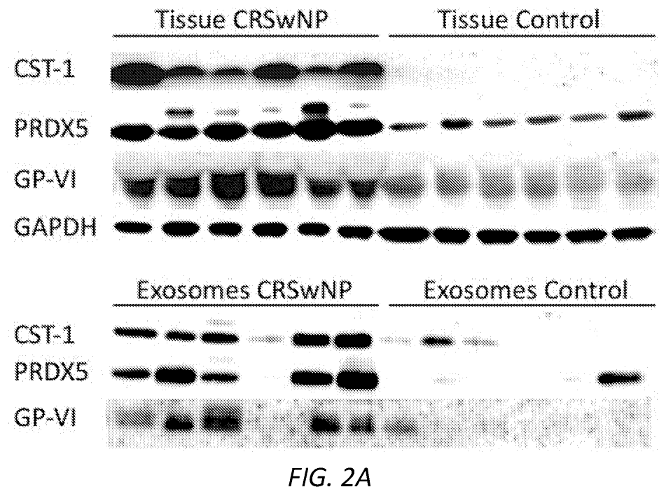

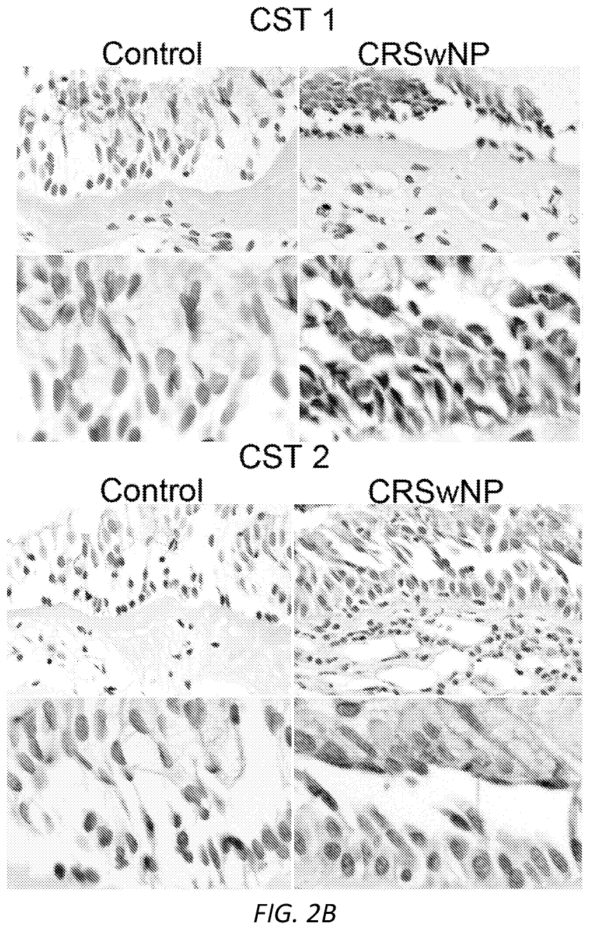

In order to validate the proteomic data set, we performed western blots on independent samples of tissue and exosomes in CRSwNP and control patients (n=6 per group) for CST1, PRDX5 and GP6. These results matched the proteomic results as CST1, PRDX5 and GP6 were significantly differentially expressed in both tissue (CST1, p<0.001; PRDX5, p<0.001; GP6, p<0.001) and exosomes (CST1, p<0.01; PRDX5, p<0.01; GP6, p<0.01) among the CRSwNP group relative to control (FIG. 2A).

Using IHC, CST-1 expression was found to be localized to the epithelial cytoplasm in CRSwNP. No significant staining was seen within the stroma or epithelial glands (see FIG. 2B).

### Example 3. Exosomal CST 1 and 2 Correlate with Tissue Expression and Clinical Parameters

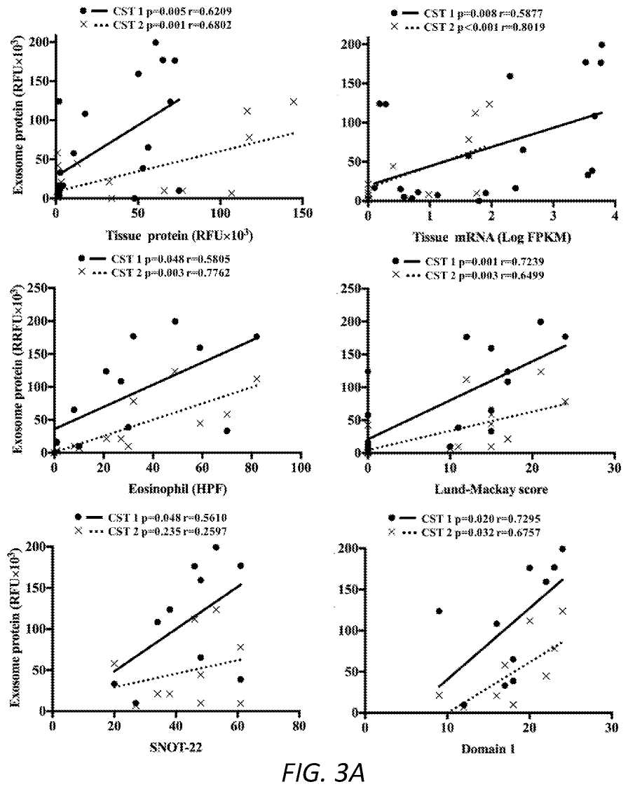

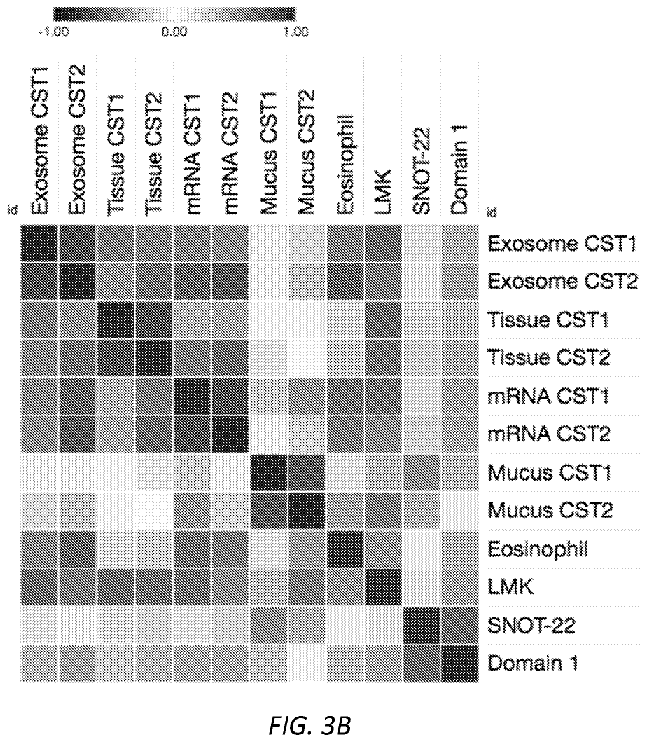

Both exosomal CST 1 and 2 exhibited a strong and significant correlation with matched tissue protein (r=0.62, p=0.005 and r=0.68, p=0.001; respectively) and mRNA expression levels (r=0.59, p=0.008 and r=0.80, p<0.001; respectively). Similarly, exosomal CST 1 and 2 strongly and significantly correlated with tissue eosinophil per hpf and Lund-Mackay Scores. Exosomal CST 1 significantly correlated with total and Domain 1 SNOT-22 scores while CST 2 correlated only with Domain 1 scores. Mucus CST1/2 protein levels tended to correlate poorly with other sample sources (e.g., exosomal protein, tissue protein and tissue mRNA) as well as with clinical disease parameters (see FIG. 3).

### Example 4. Exosomal CST 1 and 2 Associate with Allergy and AERD

We next examined the association with between exosomal CST expression, allergy, and AERD. None of the control patients (n=20) had allergy or AERD. Among the CRSwNP group, 3 out of 20 had a history of allergy or AERD, with one patient endorsing a history of both. Patients with CRSwNP with allergy demonstrated a non-significant trend towards increased exosomal CST 1 and 2 expression (mean+/−SEM 80,266+/−55,527 and 28,001+/−17,988 RFUs; respectively) relative to CRSwNP without allergy (mean+/−SEM 39,992+/−8,528 p=0.18 and 11,872+/−5,981 RFUs p=0.19; respectively). A similar trend was seen with respect to AERD (see FIG. 4).

### Example 5. CST 1 and 2 Exome Analysis

Three variants were identified in the promoter region of the CST1 transcript, as compared to the 18 variants identified in the transcript region. Similarly, 4 variants were identified in the promoter region for CST2, as compared to 16 variants identified in the transcript region. None of the variants in CST1 or CST2 transcript region are of high or moderate impact based on functional consequence checking as described in the variant prioritization section (see Table 3).

TABLE 3
CST 1 and 2 exome variants

| Chr | Start | End | Ref | Alt | Gene |
|---|---|---|---|---|---|
| chr20 | 23733434 | 23733434 | C | T | CST1 |
| chr20 | 23731589 | 23731589 | G | C | CST1 |
| chr20 | 23731560 | 23731560 | C | T | CST1 |
| chr20 | 23731494 | 23731494 | A | G | CST1 |
| chr20 | 23731426 | 23731426 | C | T | CST1 |
| chr20 | 23731412 | 23731412 | G | A | CST1 |
| chr20 | 23731110 | 23731110 | G | A | CST1 |
| chr20 | 23731033 | 23731033 | C | T | CST1 |
| chr20 | 23729722 | 23729722 | G | T | CST1 |
| chr20 | 23729555 | 23729555 | G | A | CST1 |
| chr20 | 23729555 | 23729555 | — | CA | CST1 |
| chr20 | 23729554 | 23729554 | — | AC | CST1 |
| chr20 | 23729536 | 23729536 | G | A | CST1 |
| chr20 | 23729534 | 23729534 | C | T | CST1 |
| chr20 | 23729514 | 23729514 | C | T | CST1 |
| chr20 | 23729500 | 23729500 | T | C | CST1 |
| chr20 | 23729496 | 23729496 | C | T | CST1 |
| chr20 | 23729486 | 23729486 | T | G | CST1 |
| chr20 | 23729410 | 23729410 | C | G | CST1 |
| chr20 | 23729134 | 23729134 | G | C | CST1 |
| chr20 | 23728847 | 23728847 | A | G | CST1 |
| chr20 | 23809107 | 23809107 | A | G | CST2 |
| chr20 | 23809055 | 23809055 | A | G | CST2 |
| chr20 | 23807360 | 23807360 | G | A | CST2 |
| chr20 | 23807308 | 23807308 | G | C | CST2 |
| chr20 | 23807028 | 23807028 | A | C | CST2 |
| chr20 | 23806929 | 23806929 | A | C | CST2 |
| chr20 | 23806643 | 23806643 | C | T | CST2 |
| chr20 | 23806295 | 23806295 | C | A | CST2 |
| chr20 | 23806183 | 23806183 | A | G | CST2 |
| chr20 | 23806008 | 23806008 | C | T | CST2 |
| chr20 | 23805832 | 23805832 | G | A | CST2 |
| chr20 | 23805802 | 23805802 | C | T | CST2 |
| chr20 | 23805720 | 23805720 | T | C | CST2 |
| chr20 | 23805690 | 23805690 | T | G | CST2 |
| chr20 | 23805609 | 23805609 | T | C | CST2 |
| chr20 | 23805548 | 23805548 | C | G | CST2 |
| chr20 | 23805520 | 23805520 | C | G | CST2 |
| chr20 | 23805475 | 23805475 | A | C | CST2 |
| chr20 | 23804923 | 23804923 | A | — | CST2 |
| chr20 | 23804864 | 23804864 | C | T | CST2 |

### Example 6. Th2 with Recombinant Proteins and SEB

The research protocol for this study was approved by the Massachusetts Eye and Ear Infirmary Institutional Review Board. Control ethmoid mucosal explant specimens were obtained from patients undergoing functional endoscopic sinus surgery. For each patient, 6 organotypic explants were prepared using a 5-mm biopsy punch, taking care to ensure that each contained an intact epithelial layer. The explants were exposed to the vehicle control (serum- and hydrocortisone-free bronchial epithelial growth medium; Lonza, Basel, Switzerland) for 1 hour at 37° C. and the media supernatant was then collected. The explants were then exposed to CST1 (Sinobiological) at 6.25 μg/mL or CST2 (Abcam) at 12.5 μg/mL recombinant proteins for 24 hours at 37° C. and the media supernatant was then collected. The supernatants were then exposed to IL-6, 8, and TSLP ELISAs (eBioscience, San Diego, Calif.) and Micro BCA Protein Assay Kit (Pierce, Rockford, Ill.) to quantify cytokine expression after exposure to recombinant CST1 or 2. Hour 24 was normalized by hour 1 to determine the Th2 expression. All samples were analyzed in duplicate and statistical significance was determined using the Student's t-Test when compared to the vehicle control.

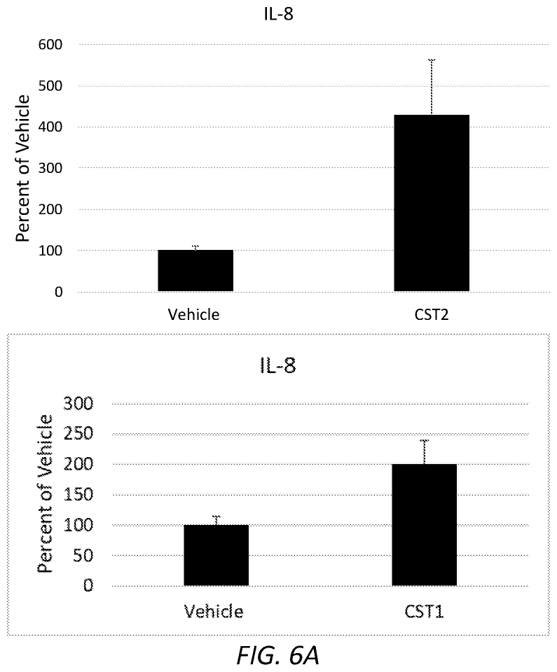

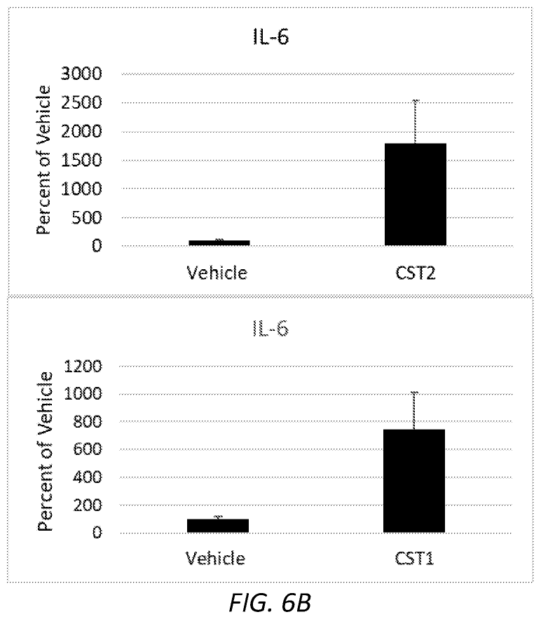

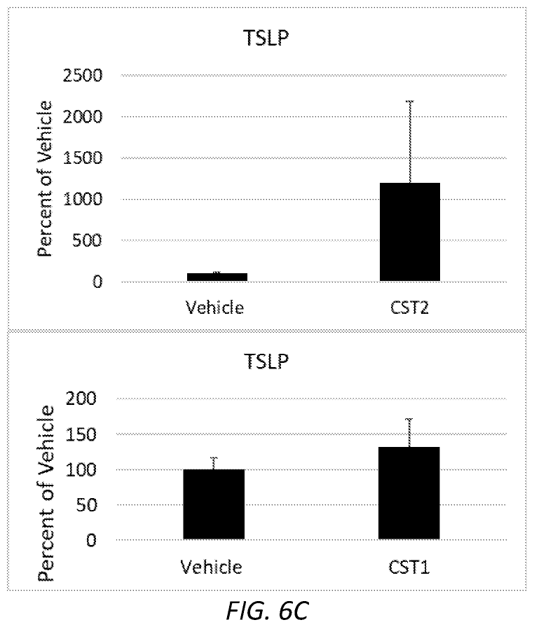

As shown in FIG. 6A, the addition of CST2 significantly increased IL-8 secretion (437.5+134.9% (Mean+SEM), p=0.03, n=5) when compared to baseline (100+11.3%) in healthy control patients. The addition of CST1 also significantly increased IL-8 secretion (200.7+38.4% (Mean+SEM), p=0.02, n=5) when compared to baseline (100+13.7%) in healthy control patients.

As shown in FIG. 6B, the addition of CST2 significantly increased IL-6 secretion (1789.1+749%, p=0.04, n=5) when compared to baseline (100+16.3%) in healthy control patients. The addition of CST1 significantly increased IL-6 secretion (742.7+271.2%, p=0.03, n=5) when compared to baseline (100+16.3%) in healthy control patients.

As shown in FIG. 6C, the addition of CST2 increased TSLP secretion (1194.2+699.5%, p=0.14, n=4) when compared to baseline (100+24.4%) in healthy control patients. The addition of CST1 increased TSLP secretion (132.7+38.8%, p=0.45, n=4) when compared to baseline (100+17.4%) in healthy control patients.

In further experiments, CRSwNP ethmoid mucosal explant specimens were obtained from patients undergoing functional endoscopic sinus surgery. For each patient, 8 organotypic explants were prepared using a 5-mm biopsy punch, taking care to ensure that each contained an intact epithelial layer. The explants were exposed to the vehicle control (serum- and hydrocortisone-free bronchial epithelial growth medium; Lonza, Basel, Switzerland) for 1 hour at 37° C. and the media supernatant was then collected. The explants were then exposed to the CST1 (R&D Systems) at 4 μg/mL or CST2 (Abcam) at 12 μg/mL recombinant proteins with or without the presence of 0.5 mg/mL SEB (Staphylococcus aureus enterotoxin B, Toxin Technology, Sarasota, Fla.) for 1 hour at 37° C. and the media supernatant was then collected. The supernatants were then exposed to IL-5, 6, 8, and TSLP ELISAs (eBioscience, San Diego, Calif.) and Micro BCA Protein Assay Kit (Pierce, Rockford, Ill.) to quantify cytokine expression after exposure to recombinant CST1 or 2. Hour 2 was normalized by hour 1 to determine the Th2 expression. All samples were analyzed in duplicate and statistical significance was determined using the Student's t-Test when compared to the vehicle control.

The results showed that stimulation of nasal polyp explants with CST 1 and 2 promote the secretion of TH2 related (e.g. polyp forming) cytokines at levels similar to those of Staphylococcus enterotoxin B.

### Example 7. Protease Inhibition

Papain (Papain from papaya latex, Sigma, St. Louis, Mo.) was used as a model protease to determine protease activity using a commercially available kit (Protease Assay Kit, EMD Millapore, Burlington, Mass.). The kit was run according to the manufacturer's instructions, with the addition of a Papain standard curve. After determining the optimal dose of Papain (0.625 and 0.3125 mg/mL), recombinant CST1 (R&D Systems, Minneapolis, Minn.) or CST2 (Abcam, Cambridge, Mass.) was added to the determine if the Papain protease activity could be inhibited. The recombinant cystatins were incubated with Papain at 37° C. for 30 minutes. The optimal dose for recombinant CST1 was determined to be 12.5 μg/mL and 6.25 μg/mL for CST2. To determine if the protease inhibition could be abrogated, monoclonal antibodies for CST1 (R&D Systems) or CST2 (Mybiosource, San Diego, Calif.) were incubated with the recombinant proteins at 37° C. for 30 minutes, and then Papain was added to the mixture and incubated at 37° C. for 30 minutes. All samples were analyzed in duplicate and statistical significance was determined using the Student's t-Test when compared to the papain alone or papain with recombinant protein.

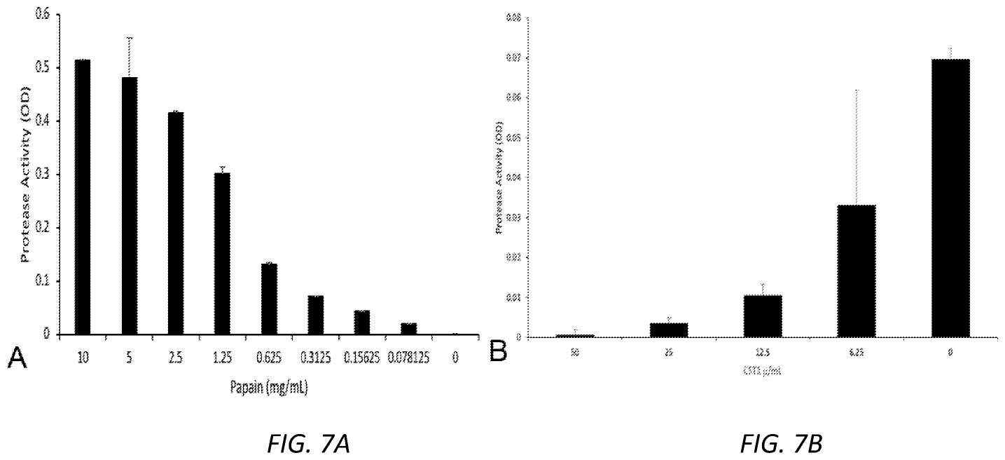

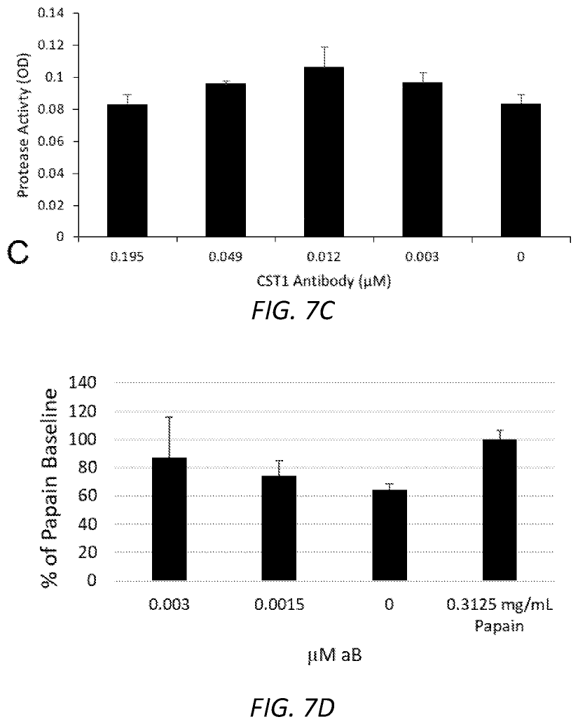

The results showed that protease activity significantly increased in a dose response manner when the papain concentration increased (p<0.01, n=1) (FIG. 7A). Protease activity was significantly reduced when recombinant CST1 was added to papain at 50 μg/mL (0.0005±0.0014 OD (Optical Density, Mean±Standard Deviation), p=0.001, n=1), 25 μg/mL (0.0035±0.0014 OD, p=0.001, n=1), and 12.5 μg/mL (0.011±0.0028 OD, p=0.002, n=1), but not at 6.25 μg/mL (0.033±0.0029 OD, p=0.2, n=1), when compared to papain only (0.07±0.0028 OD, n=1) (FIG. 7B).

The addition of various concentrations of anti-Human CST1 antibody to 0.625 mg/mL papain and 12.5 μg/mL recombinant CST1 increased protease activity (0.195 μM, 0.07±0.016 OD, p=0.85; 0.049 μM, 0.079±0.019 OD, p=0.38; 0.012 μM, 0.08±0.019 OD, p=0.53; 0.003 μM, 0.075±0.025 OD, p=0.6; when compared to papain and recombinant CST1 alone (0.067±0.019 OD, n=2) but not significantly, as shown in FIG. 7C.

As shown in FIG. 7D, 6.25 μg/mL recombinant CST2 significantly decreased protease activity (64.3±4.3%, p=0.03, n=2) when compared to 0.3125 mg/mL papain (100±6.4%, n=2). The addition of an anti-Human CST2 antibody to 6.25 μg/mL recombinant CST2 to 0.3125 mg/mL papain also increased protease activity. Protease activity increased at 0.003 μM (87.4±28.6% of Baseline, p=0.06, n=2) and significantly increased at 0.0015 μM (74.5±10.3%, p=0.02, n=2) anti-Human CST2 antibody when compared to 0 (6.25 μg/mL recombinant CST2 to 0.3125 mg/mL papain, 64.3±4.3%, n=2).

### Example 8. Th2 Inflammatory Analysis with Recombinant CST1/2 Proteins and Antibodies

The research protocol for this study was approved by the Massachusetts Eye and Ear Infirmary Institutional Review Board. CRSwNP ethmoid mucosal explant specimens were obtained from patients undergoing functional endoscopic sinus surgery. For each patient, 6 organotypic explants were prepared using a 5-mm biopsy punch, taking care to ensure that each contained an intact epithelial layer. The explants were exposed to the vehicle control (serum- and hydrocortisone-free bronchial epithelial growth medium; Lonza, Basel, Switzerland) for 1 hour at 37° C. and the media supernatant was then collected. The explants were then exposed to the CST1 (R&D Systems) at 12.5 μg/mL recombinant protein with or without the presence of 0.195 mM CST1 antibody (Human Cystatin SN Antibody, R&D Systems) for 1 hour at 37° C. and the media supernatant was then collected. The supernatants were then exposed to IL-5, 6, 8, and TSLP ELISAs (eBioscience, San Diego, Calif.) and Micro BCA Protein Assay Kit (Pierce, Rockford, Ill.) to quantify cytokine expression after exposure to recombinant CST1 or 2. Hour 24 was normalized by hour 1 to determine the Th2 expression. All samples were analyzed in duplicate and statistical significance was determined using the Student's t-Test when compared to the vehicle control.

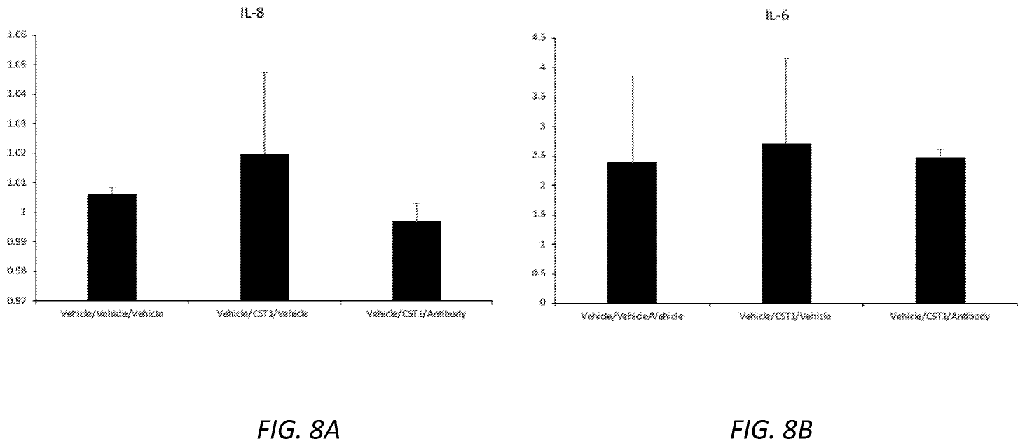

As shown in FIG. 8A, IL-8 secretion increased with addition of CST1 (1.02±0.03, p=0.6, n=1), when compared to the vehicle control (1.01±0.02) in patients with CRSwNP. After the addition of the antibody IL-8 secretion decreased (0.997±0.006) when compared to both the vehicle control (1.01±0.02, p=0.2) and CST1 alone (1.02±0.03, p=0.4). As shown in FIG. 8B, IL-6 secretion increased with addition of CST1 (2.71±1.45, p=0.85, n=1), when compared to the vehicle control (2.4±1.46) in patients with CRSwNP. After the addition of the antibody IL-8 secretion decreased (2.47±0.14) when compared to both the vehicle control 2.4±1.46, p=0.95) and CST1 alone (2.71±1.45, p=0.85).

### Example 9. Discriminant Analysis Followed by Unsupervised Cluster Analysis Including Exosomal Cystatins Predict Presence of Chronic Rhinosinusitis, Phenotype, and Disease Severity

Despite recent efforts in determining specific subcategories of CRS⁷, optimal diagnostic and treatment algorithms remain elusive due to the complex etiopathology of the CRS, its variability between populations^{8,9,10}, and the need for invasive tissue sampling to establish endotypes¹¹.

Unsupervised cluster analysis (UCA) seeks to enable the sorting of CRS patients into groups based on an array of clinical and biologic variables in order to identify novel categories which may inform intrinsic disease severity and prognosis¹². Previous studies have been successful in applying this technique based on tissue derived cytokines or biomarkers^{11,13,14,15,16,17}. However, at present, these cluster results remain inconsistent and limited by the need to performed invasive biopsies either in clinic or at the time of surgery.¹⁸

Cystatins (CST) are endogenous protease inhibitors that play key roles in epithelial barrier and immunomodulatory processes. It has been hypothesized that cystatins may contribute to the recurrence of eosinophilic CRS and allergic rhinitis by interacting with various factors such as Th2 cytokines, infection, fibroblasts²⁵ and inhibition of allergen-related histamine release²¹. Cystatin-SN (CST-1) has been described as being both transiently elevated in seasonal allergic rhinitis^{21,22,23} and tonically overexpressed in the setting of eosinophilic CRS.²⁴ Similarly, recent studies have associated high cystatin-SA (CST-2) levels with severe and steroid resistant asthma^{26,27}. In related work, CST-2 adhesion to human fibroblasts has been associated with the enhancement of NF-kB activity²⁸ suggesting cystatins as important contributors to a range of respiratory inflammatory diseases.

Exosomes are 30- to 150-nm^{29,30} vesicles secreted by virtually all cell types into multiple body fluids and can be isolated from nasal mucus in both healthy and CRS patients.^{31,32,33,34} Exosomes contain proteins, lipids, DNA, mRNA and microRNA specific to their cell of origin and reflective of their pathophysiologic state^{35,36,37,38}. As a result of these unique properties, our group has shown that nasal mucus derived exosomes (NMDEs) proteomic analysis can yield novel non-invasive biosignatures that outperform whole mucus sampling with respect to signal-to-noise ratio and predictive power for the presence of CRS with Nasal Polyps (CRSwNP).^{33,38,39,40,41} Furthermore, we have shown that NMDE Cystatin levels are profoundly elevated in highly selected CRSwNP patients relative to healthy controls⁴⁰.

Given the confluence of the previously described findings, the purpose of this study was to utilize NMDE CST-1 and CST-2 as biomarkers across a large, heterogenous patient population to determine if they could be used to predict disease, phenotype, and severity using a non-invasive, mucus sampling protocol.

Methods

Study Design

Clinical data and mucus sampling were approved by the Institutional Review Board of Massachusetts Eye and Ear (Protocol #196461-47). In total, 105 subjects undergoing a planned sinonasal procedure were recruited. Patients were diagnosed as CRSsNP (n=33) or CRSwNP (n=40) by the International Consensus Statement on Allergy and Rhinology (ICAR:RS) criteria⁴². Healthy patients (e.g. controls) were considered those undergoing surgery for non-inflammatory disease (n=32). Exclusion criteria included patients with ciliary dysfunction, autoimmune disease, cystic fibrosis, or immunodeficiency. Among controls, additional exclusion criteria were presence of current smoking or asthma. Diagnostic criteria for allergy were based on both clinical history and clinical testing (by either RAST or skin prick). Control subjects with allergic rhinitis were not excluded. All subjects were aged 18 to 79 years. The indications for surgery and its procedures were based on clinical decisions independent from participation in the study.

Mucus and Exosome Collection Technique

Mucus samples were collected at the beginning of surgery by applying a total of 8 compressed polyvinyl alcohol (PVA) sponges (4 per side, Medtronic, Minneapolis, Minn.) into bilateral nasal cavities taking care not to abrade the mucosa. Any sponge visually contaminated with blood was excluded. Each sponge was removed after 5 minutes, placed individually in a microcentrifuge tube and immediately frozen at −80° C. for further analysis.

Exosome Purification Technique from Whole Mucus

The exosome purification technique was adapted from the ultracentrifugation (UCF) procedure described by Thery et al³⁴ and reported by our group previous studies^{39,43,41}. Mucus samples were extracted from the PVA sponges by centrifugation (1500 g at 4° C. for 30 minutes and then 3000 g at 4° C. for 10 minutes). The mucus from all sponges of the same patient was pooled, transferred to a new tube, and then diluted to 1.5 ml total with 1×phosphate-buffered saline (PBS; Life Technologies, Carlsbad, Calif.). Cellular debris were pelleted by centrifugation for 45 minutes at 12,000 g at 4° C. The supernatant was then suspended in 1.5 mL 1×PBS in UCF tubes (Thinwall, 2 ml; Thermo Scientific, Waltham, Mass.) and ultracentrifuged for 2 hours at 110,000 g, at 4° C. The supernatant was collected and the pellet was resuspended in 1.5 mL 1×PBS. The suspension was filtered through a 0.22-μm filter (Fisher Scientific, Pittsburgh, Pa.) and collected in the UCF tube. The filtered suspension was then centrifuged for 70 minutes at 110,000 g at 4° C. The supernatant was collected and the pellet was resuspended in 800 μL M-per (M-PER Mammalian Protein Extraction Reagent, Thermo Scientific) with protease inhibitor (Protease Inhibitor Cocktail, Sigma Aldrich, St. Louis, Mo.). The resuspended pellet was then incubated for 15 minutes at 37° C. and later centrifuged for 5 minutes at 14000 g. The lysate supernatant was transferred to a new microcentrifuge tube and frozen at −80° C. for further analysis.

Cystatin Quantification in Purified NMDEs

The purified exosome lysate from all patients was subjected to Cystatin-SA (CST-2) and Cystatin-SN (CST-1) ELISAs (Mybiosource, San Diego, Calif.) to determine the relative concentration within the purified exosomal fraction. All values were normalized to the total protein concentration within the same sample using a Micro BCA Protein Assay Kit (Pierce, Rockford, Ill.).

Statistical Analysis

Stata version 13 (StataCorp, College Station, Tex.) software was used for unsupervised hierarchical cluster analysis of mixed (continuous and binomial) data, performed using Ward's linkage technique. This was followed by application of Duda/Hart Je(2)/Je(1) index cluster stopping rules, maximizing Je(2)/Je(1) and minimizing pseudo T-squared to determine the optimal number of clusters described by the data. Large Je(2)/Je(1) values indicate more distinct clustering, while small pseudo-T-squared values indicate more distinct clustering. The variables included in the cluster analysis were normalized values of Cystatin-SN and Cystatin-SA collected in Purified NMDEs, diagnosis (Control, CRSsNP, CRSwNP), age, gender, race, active allergy (perennial and in season allergy), asthma, NSAID allergy and presence of eosinophilia on pathologic analysis. Non-biological variables, including SNOT-22 scores and surgical history, were not included in the cluster analysis. Once clusters were generated, individual cluster characteristics were evaluated utilizing ANOVA for continuous variables with equal variance, Welch's test for continuous variables with unequal variance, and Fisher's exact test for categorical variables. Multidimensional Scaling was applied to subject data to visualize cluster dissimilarities in two dimensions.

For all other statistical analysis, Stata version 13 (StataCorp, College Station, Tex.) and GraphPad Prism version 8.0.2 (GraphPad Software, La Jolla, Calif. USA) softwares were utilized to assess differences in patient demographics as well as in protein levels. Differential protein expression in between phenotype groups was calculated using the Student t test. A p value of <0.05 was considered statistically significant.

Results

Patient Demographics

Demographic and phenotype data is tabulated in Table 4. There were no significant differences between groups with respect to age, race, active and environmental allergy, SNOT-22 (score and domains) and use of medication in the past month. Asthma, NSAID allergy, Caucasian ethnicity, and the presence of tissue eosinophilia were significantly higher in the CRSwNP group as compared to one or both of the Control and CRSsNP groups.

TABLE 4
Patient Demographics and Clinical Characteristics

| | | | | | | One-way |
|---|---|---|---|---|---|---|
| | CRSwNP | CRSsNP | | Control | | ANOVA |
| Variable | (n = 40) | (n = 33) | | (n = 32) | | p value |
| Age, in years (mean ± SD) | 49.4 ± 14.9 | 46.7 ± 15.3 | | 41.3 ± 16.9 | | 0.986 |
| Gender [n (%)] | | | | | | |
| Male | 31 (77.5%) | 13 | (39.4%) | 18 | (56.3%) | 0.003* |
| Race [n (%)] | | | | | | |
| Caucasian | 37 (92.5%) | 26 | (78.8%) | 24 | (75.0%) | 0.113 |
| Smoking [n (%)] | | | | | | |
| Previous smoker | 15 (37.5%) | 11 | (33.3%) | 3 | (9.4%)** | 0.028*^{,}** |
| Allergy [n (%)] | | | | | | |
| Active | 23 (57.5%) | 13 | (39.4%) | 13 | (40.6%) | 0.221 |
| Environmental | 25 (62.5%) | 14 | (42.4%) | 13 | (40.6%) | 0.114 |
| Asthma | 30 (75.0%) | 8 | (24.2%) | 0 | (0%)** | <0.0001* |
| NSAID | 11 (27.5%) | 0 | (0%) | 1 | (3.1%) | 0.0002* |
| Tissue Eosinophilia [n (%)] | 32 (80.0%) | 7 | (21.2%) | 1 | (3.1%) | <0.0001* |
| SNOT-22 Score (mean ± SD) | 39.6 ± 26.8 | 38.2 ± 17.0 | | 38.2 ± 25.9 | | 0.965 |
| Domain 1 | 16.4 ± 8.1 | 11.8 ± 5.5 | | 13.0 ± 8.5 | | 0.051 |
| Domain 2 | 5.9 ± 4.5 | 5.4 ± 3.6 | | 4.6 ± 4.6 | | 0.359 |
| Domain 3 | 5.7 ± 4.9 | 7.2 ± 4.6 | | 6.4 ± 5.8 | | 0.371 |
| Domain 4 | 10.1 ± 11.1 | 11.5 ± 9.4 | | 11.3 ± 10.4 | | 0.682 |
| Domain 5 | 9.3 ± 8.6 | 10.7 ± 6.9 | | 10.8 ± 8.6 | | 0.549 |
| Previous Sinus Surgery [n (%)] | 19 (47.5%) | 9 | (27.3%) | 2 | (6.3%) | 0.0004* |
| Depression [n (%)] | 2 (4.9%) | 7 | (21.2%) | 5 | (12.8%) | <0.0001* |
| Medication use (Past Month) | | | | | | |
| Systemic Steroids [n (%)] | 6 (15.0%) | 1 | (3.0%) | 1 | (3.1%) | 0.083 |
| Topical Steroids [n (%)] | 23 (57.5%) | 15 | (45.5%) | 16 | (50.0%) | 0.588 |
| Antibiotics [n (%)] | 2 (5.0%) | 4 | (12.1%) | 3 | (9.4%) | 0.554 |
| Antihistamines [n (%)] | 15 (37.5%) | 12 | (36.4%) | 7 | (21.9%) | 0.318 |

| | | | | |
|---|---|---|---|---|
| CST1 (mean ± SEM) | 360.0 ± 49.3 | 65.1 ± 5.8 | 349.4 ± 35.1 | <0.0001* |
| CST2 (mean ± SEM) | 130.5 ± 16.7 | 56.6 ± 8.3 | 23.4 ± 4.1 | <0.0001* |

Exosomal Biomarkers

Normalized exosomal cystatin data by phenotype is tabulated in Table 4. The normalized values of CST-1 in CRSwNP patients (360.0±49.3 mean±SEM pg/μg; n=40) and in Control patients (349.4±35.1; n=32) were significantly higher (p<0.0001) than in the CRSsNP group (65.1±5.8; n=33). Expression of CST-2 in the CRSwNP group (130.5±16.7 mean±SEM pg/μg; n=40) was significantly higher (p<0.0001) than in both CRSsNP (56.6±8.3; n=33) and Control groups (23.4±4.2; n=32). The logarithmic scatterplot of CST-1 versus CST-2 values provided a qualitative trend towards segregation by phenotype.

Cluster Analysis

Unsupervised clustering of all patients based on cystatin quantification, diagnosis, demographic and biological variables, resulted in an optimal outcome of 7 clusters after application of stopping rules. The differentiation was reflected directly in clinical phenotypes, with 2 clusters (1 and 2) corresponding to controls, 2 clusters (3 and 4) to CRSsNP and 3 clusters (5,6 and 7) to CRSwNP. Age did not differ significantly between groups (p=0.3) however, all the other variables presented important and significant differences between clusters (p<0.001, Table 5).

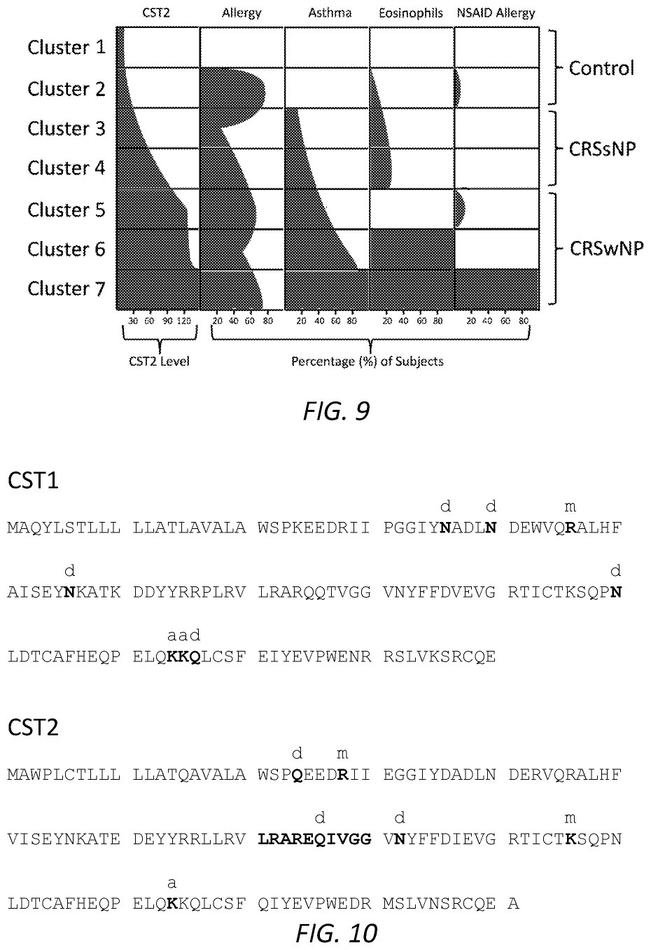

Clusters and associated biological variables are displayed in FIG. 9. CST-2 levels represented one of the most important differentiating variables between clusters which trended linearly with phenotype and clinical severity parameters.

Clusters 1 and 2 presented predominantly with low CST-2 values and were comprised of control subjects. These clusters had a similar proportion of patients and while cluster 1 was exclusively composed of Caucasians with no active allergy, cluster 2 had a higher prevalence of allergy.

Intermediate-expression of CST-2 corresponded to the CRSwNP groupings (clusters 3 and 4). Both clusters also had a similar proportion of patients and could further be differentiated by the higher prevalence of active allergy in cluster 4, comprised exclusively of non-Caucasian females.

The latter 3 clusters were characterized by high CST2 expression. These clusters consisted of CRSwNP patients and could further be differentiated as follows: cluster 5 corresponded to a non-eosinophilic inflammatory phenotype; cluster 6 corresponded to the eosinophilic subjects without NSAID allergy; and cluster 7 was comprised entirely of AERD patients with asthma and eosinophilia.

Multidimensional scaling techniques allow for visualization of differences between subjects in a two-dimensional space, with cluster assignments highlighted. Dissimilarities between subjects across all clustering variables are directly reflected by distance on these axes.

TABLE 5
Clinical variables by patient cluster

| | Total | | Cluster 1 | | Cluster 2 | | Cluster 3 | | Cluster 4 | |
|---|---|---|---|---|---|---|---|---|---|---|
| Patients (N) | 105 | | 15 | | 17 | | 16 | | 17 | |
| Cluster Variables | | | | | | | | | | |
| CTRL | 32 | (30%) | 15 | (100%) | 17 | (100%) | 0 | (0%) | 0 | (0%) |
| CRSsNP | 33 | (31%) | 0 | (0%) | 0 | (0%) | 16 | (100%) | 17 | (100%) |
| CRSwNP | 40 | (38%) | 0 | (0%) | 0 | (0%) | 0 | (0%) | 0 | (0%) |
| CST1 | 264.1 | (258.7) | 375.6 | (254.5) | 326.2 | (135.8) | 61.3 | (32.1) | 68.5 | (35.4) |
| CST2 | 74.7 | (84.6) | 21.9 | (15.9) | 24.8 | (29.8) | 46.1 | (34.3) | 66.5 | (57.2) |
| PAPPA | 71.2 | (72.0) | 100.9 | (55.6) | 80.4 | (28.9) | 59.4 | (31.5) | 64.9 | (29.9) |
| Age | 46.0 | (15.8) | 44.6 | (17.0) | 38.5 | (16.7) | 46.4 | (12.7) | 47 | (17.8) |
| Gender (Female) | 43 | (41%) | 5 | (33%) | 9 | (53%) | 3 | (19%) | 17 | (100%) |
| Race (Non-White) | 18 | (17%) | 0 | (0%) | 8 | (47%) | 7 | (44%) | 0 | (0%) |
| Active Allergy | 49 | (47%) | 0 | (0%) | 13 | (76%) | 4 | (25%) | 9 | (53%) |
| Asthma | 38 | (36%) | 0 | (0%) | 0 | (0%) | 3 | (19%) | 5 | (29%) |
| NSAID Allergy | 12 | (11%) | 0 | (0%) | 1 | (6%) | 0 | (0%) | 0 | (0%) |
| Path Eosinophils | 40 | (38%) | 0 | (0%) | 1 | (6%) | 3 | (19%) | 4 | (24%) |
| Non-Cluster Variables | | | | | | | | | | |
| PrevSurg | 30 | (29%) | 2 | (13%) | 0 | (0%) | 1 | (6%) | 8 | (47%) |
| SNOT-22 | 37.8 | (23.7) | 34.1 | (29.6) | 38.6 | (25.4) | 39.2 | (15.7) | 37.2 | (18.8) |
| Domain 1 | 13.7 | (7.8) | 10.7 | (9.3) | 13.4 | (8.4) | 12.5 | (5.9) | 11.1 | (5.3) |
| Domain 2 | 5.2 | (4.3) | 2.5 | (4.2) | 5.4 | (4.6) | 5.9 | (4.1) | 4.9 | (3.1) |
| Domain 3 | 6.1 | (4.9) | 5.4 | (7.1) | 5.5 | (3.8) | 6.5 | (4.6) | 7.8 | (4.6) |
| Domain 4 | 10.6 | (10.3) | 11.3 | (12.1) | 11.4 | (10.8) | 11.9 | (8.2) | 11 | (10.7) |
| Domain 5 | 10.0 | (8.1) | 10.4 | (9.8) | 11.1 | (8.8) | 11.1 | (6.5) | 10.3 | (7.5) |

| | | | |
|---|---|---|---|
| Cluster 5 | Cluster 6 | Cluster 7 | p-value |

| | | | |
|---|---|---|---|
| Patients (N) | 8 | 22 | 10 |

| | | | | | | | |
|---|---|---|---|---|---|---|---|
| Cluster Variables | | | | | | | |
| CTRL | 0 | (0%) | 0 | (0%) | 0 | (0%) | <0.001 |
| CRSsNP | 0 | (0%) | 0 | (0%) | 0 | (0%) | <0.001 |
| CRSwNP | 8 | (100%) | 22 | (100%) | 10 | (100%) | <0.001 |
| CST1 | 488.9 | (529.4) | 360.7 | (211.9) | 255.5 | (268.5) | <0.001 |
| CST2 | 122.7 | (131.3) | 126.8 | (97.0) | 144.9 | (111.6) | <0.001 |
| PAPPA | 121.5 | (155.5) | 38.5 | (42.6) | 73.1 | (143.5) | 0.02 |
| Age | 52.4 | (17.9) | 50.5 | (14.6) | 44.4 | (13.2) | 0.3 |
| Gender (Female) | 0 | (0%) | 6 | (27%) | 3 | (30%) | <0.001 |
| Race (Non-White) | 0 | (0%) | 2 | (9%) | 1 | (10%) | <0.001 |
| Active Allergy | 5 | (63%) | 11 | (50%) | 7 | (70%) | <0.001 |
| Asthma | 4 | (50%) | 16 | (73%) | 10 | (100%) | <0.001 |
| NSAID Allergy | 1 | (13%) | 0 | (0%) | 10 | (100%) | <0.001 |
| Path Eosinophils | 0 | (0%) | 22 | (100%) | 10 | (100%) | <0.001 |
| Non-Cluster Variables | | | | | | | |
| PrevSurg | 2 | (25%) | 10 | (45%) | 7 | (70%) | <0.001 |
| SNOT-22 | 41.5 | (25.6) | 32.3 | (25.0) | 50.1 | (27.6) | 0.72 |
| Domain 1 | 17.1 | (6.5) | 14.2 | (8.0) | 19.8 | (8.5) | 0.12 |
| Domain 2 | 7.5 | (4.6) | 4.1 | (4.1) | 8 | (4.1) | 0.06 |
| Domain 3 | 5.4 | (5.6) | 4.4 | (3.9) | 9.4 | (5.5) | 0.21 |
| Domain 4 | 10.1 | (12.7) | 8.5 | (10.1) | 11.1 | (9.5) | 0.98 |
| Domain 5 | 9.5 | (8.3) | 7.3 | (8.1) | 12 | (8.8) | 0.78 |

Discussion

Current guidelines stratify CRS into phenotypes that fail to capture the diverse pathophysiology and immunologic profiles underlying sinonasal inflammation^{6,1}. Despite robust efforts in unraveling specific endotypes and individualizing therapies⁷, optimal diagnostic and treatment algorithms remain elusive.^{8,9,10} There persists an urgent need to develop better tools to identify and predict CRS endotypes. These efforts, in turn, promise to provide new insights into the mechanisms driving inflammation and reveal potential novel therapeutic targets.

The goal of this study was to therefore utilize a study design combining a UCA approach with established non-invasive and sensitive biomarkers of CRS³⁹ to determine whether NMDE CSTs could be used to both predict disease and classify patients into different subgroups. The UCA removes any a priori biases and allows for an overview of multiple variables thereby revealing new or unexpected groupings¹². This approach has been reported previously using clinical and tissue based parameters^{11,15,16,17} however these approaches required invasive sampling and therefore could not be used prospectively.

While both our group and others have reported on a variety of novel exosomal derived biomarkers in CRS³⁹, we elected to focus on CST-1 and 2 as these appear to provide direct pathophysiologic links between allergic rhinitis and CRS and play key roles in epithelial barrier function^{21,25}. An unexpected result of our approach was that the combination of CST-1 and CST-2 provided a 2-dimensional axis upon which controls, CRSsNP, and CRSwNP could be clearly resolved. Furthermore, the addition of CST-2 to the cluster analysis revealed distinct subgroups within the CRS populations which tracked closely with other clinical variables classically predictive of disease severity including the presence of allergy, asthma, and AERD. It remains a topic of further research as to whether these groups represent distinct clinical entities with different disease trajectories and/or differential responses to treatment.

There were several limitations of our study which bear discussion. First, this analysis is subject to the same limitations as any UCA in that cluster grouping is heavily dependent on the membership and the variables included in the data.¹¹. Thus, unsupervised clustering methods offer an insight into patterns, but not necessarily a detailed comparison among groups. Second, although controls were assigned by clinical presentation, it is possible that some had early underlying subclinical CRS disease states which could influence their association with the analyte clusters. This may explain why some of the control subjects were found to have eosinophils on pathology and an isolated allergy to NSAIDs.

Conclusion

Our findings confirm that CST-1 and 2 levels in NMDEs are capable of discerning healthy controls from CRS patients with and without nasal polyps. Furthermore, unsupervised cluster analysis using NMDE CST-2 identified several CRS subgroups which tracked closely with classic markers of disease severity. As a “liquid biopsy”, non-invasive NMDE collection offers a powerful new opportunity to study disease pathophysiology, discriminate disease state, and potentially reveal novel therapeutic targets.

### REFERENCES FOR EXAMPLE 9

1. Fokkens W J, Lund V J, Mullol J, et al. European Position Paper on Rhinosinusitis and Nasal Polyps 2012. Rhinol Suppl. 2012. doi:10.4193/Rhino50E2
2. Yan C H, Hahn S, McMahon D, et al. Nitric oxide production is stimulated by bitter taste receptors ubiquitously expressed in the sinonasal cavity. Am J Rhinol Allergy. 2017. doi:10.2500/ajra.2017.31.4424
3. Blackwell D L, Lucas J W, Clarke T C. Summary health statistics for U.S. Adults: national health interview survey, 2012. Natl Cent Heal Stat Vital Heal stats. 2014. doi:24819891
4. Rudmik L. Economics of Chronic Rhinosinusitis. Curr Allergy Asthma Rep. 2017. doi:10.1007/s11882-017-0690-5
5. Smith K A, Orlandi R R, Rudmik L. Cost of adult chronic rhinosinusitis: A systematic review. Laryngoscope. 2015. doi:10.1002/lary.0.25180
6. Akdis C A, Bachert C, Cingi C, et al. Endotypes and phenotypes of chronic rhinosinusitis: A PRACTALL document of the European Academy of Allergy and Clinical Immunology and the American Academy of Allergy, Asthma & Immunology. J Allergy Clin Immunol. 2013. doi: 10.1016/j.jaci.2013.02.036
7. Dennis S K, Lam K, Luong A. A Review of Classification Schemes for Chronic Rhinosinusitis with Nasal Polyposis Endotypes. Laryngoscope Investig Otolaryngol. 2016. doi:10.1002/lio2.32
8. Kim J W, Hong S L, Kim Y K, Lee C H, Min Y G, Rhee C S. Histological and immunological features of non-eosinophilic nasal polyps. Otolaryngol—Head Neck Surg. 2007. doi:10.1016/j.otohns.2007.07.036
9. Baba S, Kagoya R, Kondo K, Suzukawa M, Ohta K, Yamasoba T. T-cell phenotypes in chronic rhinosinusitis with nasal polyps in Japanese patients. Allergy, Asthma Clin Immunol. 2015. doi:10.1186/s13223-015-0100-2
10. Zhang N, Van Zele T, Perez-Novo C, et al. Different types of T-effector cells orchestrate mucosal inflammation in chronic sinus disease. J Allergy Clin Immunol. 2008. doi:10.1016/j.jaci.2008.07.008
11. Tomassen P, Vandeplas G, Van Zele T, et al. Inflammatory endotypes of chronic rhinosinusitis based on cluster analysis of biomarkers. J Allergy Clin Immunol. 2016. doi:10.1016/j.jaci.2015.12.1324
12. Soler Z M, Hyer J M, Ramakrishnan V, et al. Identification of chronic rhinosinusitis phenotypes using cluster analysis. Int Forum Allergy Rhinol. 2015. doi:10.1002/alr.21496
13. Kim J W, Huh G, Rhee C S, et al. Unsupervised cluster analysis of chronic rhinosinusitis with nasal polyp using routinely available clinical markers and its implication in treatment outcomes. Int Forum Allergy Rhinol. 2019. doi:10.1002/alr.22221
14. Soler Z M, Hyer J M, Rudmik L, Ramakrishnan V, Smith T L, Schlosser R J. Cluster analysis and prediction of treatment outcomes for chronic rhinosinusitis. J Allergy Clin Immunol. 2016. doi:10.1016/j.jaci.2015.11.019
15. Divekar R, Rank M, Squillace D, Kita H, Lal D. Unsupervised network mapping of commercially available immunoassay yields three distinct chronic rhinosinusitis endotypes. Int Forum Allergy Rhinol. 2017. doi:10.1002/alr.21904
16. Liao B, Liu J X, Li Z Y, et al. Multidimensional endotypes of chronic rhinosinusitis and their association with treatment outcomes. Allergy Eur J Allergy Clin Immunol. 2018; 73(7):1459-1469. doi:10.1111/all.13411
17. Turner Hi, Chandra R K, Li P, Bonnet K, Schlundt D G. Identification of clinically relevant chronic rhinosinusitis endotypes using cluster analysis of mucus cytokines. J Allergy Clin Immunol. 2018. doi:10.1016/j.jaci.2018.02.002
18. Platt M P, Soler Z M, Kao S Y, Metson R, Stankovic K M. Topographic gene expression in the sinonasal cavity of patients with chronic sinusitis with polyps. Otolaryngol—Head Neck Surg. 2011. doi:10.1177/0194599811402030
19. Lalmanach G, Saidi A, Marchand-Adam S, Lecaille F, Kasabova M. Cysteine cathepsins and cystatins: From ancillary tasks to prominent status in lung diseases. Biol Chem. 2015. doi:10.1515/hsz-2014-0210
20. Dickinson D P. Salivary (SD-type) cystatins: Over one billion years in the making—But to what purpose? Crit Rev Oral Biol Med. 2002. doi:10.1177/154411130201300606
21. Imoto Y, Tokunaga T, Matsumoto Y, et al. Cystatin SN Upregulation in Patients with Seasonal Allergic Rhinitis. PLoS One. 2013. doi:10.1371/journal.pone.0067057
22. Fukuoka A, Matsushita K, Morikawa T, et al. Human cystatin SN is an endogenous protease inhibitor that prevents allergic rhinitis. Journal of Allergy and Clinical Immunology. 2018.
23. Ghafouri B, Irander K, Lindbom J, Tagesson C, Lindahl M. Comparative proteomics of nasal fluid in seasonal allergic rhinitis. J Proteome Res. 2006. doi:10.1021/pr050341h
24. Kouzaki H, Matsumoto K, Kikuoka H, et al. Endogenous protease inhibitors in airway epithelial cells contribute to eosinophilic chronic rhinosinusitis. Am J Respir Crit Care Med. 2017. doi:10.1164/rccm.201603-0529OC
25. Kato Y, Takabayashi T, Sakashita M, et al. Expression and functional analysis of CST1 in intractable nasal polyps. Am J Respir Cell Mol Biol. 2018. doi:10.1165/rcmb.2017-0325OC
26. Kuo C H S, Pavlidis S, Loza M, et al. A transcriptome-driven analysis of epithelial brushings and bronchial biopsies to define asthma phenotypes in U-BIOPRED. Am J Respir Crit Care Med. 2017. doi:10.1164/rccm.201512-2452OC
27. Singhania A, Wallington J C, Smith C G, et al. Multitissue transcriptomics delineates the diversity of airway T cell functions in asthma. Am J Respir Cell Mol Biol. 2018. doi:10.1165/rcmb.2017-0162OC
28. Kato T, Imatani T, Minaguchi K, Saitoh E, Okuda K. Salivary cystatins induce interleukin-6 expression via cell surface molecules in human gingival fibroblasts. Mol Immunol. 2002. doi:10.1016/S0161-5890(02)00144-X
29. Raposo G, Stoorvogel W. Extracellular vesicles: Exosomes, microvesicles, and friends. J Cell Biol. 2013. doi:10.1083/jcb.201211138
30. Simons M, Raposo G. Exosomes—vesicular carriers for intercellular communication. Curr Opin Cell Biol. 2009. doi:10.1016/j.ceb.2009.03.007
31. Nocera A L, Miyake M M, Seifert P, Han X, Bleier B S. Exosomes mediate interepithelial transfer of functional P-glycoprotein in chronic rhinosinusitis with nasal polyps. Laryngoscope. 2017; 127 (9):E295-E300. doi:10.1002/lary.26614
32. Wu G, Yang G, Zhang R, et al. Altered microRNA expression profiles of extracellular vesicles in nasal mucus from patients with allergic rhinitis. Allergy, Asthma Immunol Res. 2015. doi:10.4168/aair.2015.7.5.449
33. Choi E B, Hong S W, Kim D K, et al. Decreased diversity of nasal microbiota and their secreted extracellular vesicles in patients with chronic rhinosinusitis based on a metagenomic analysis. Allergy Eur J Allergy Clin Immunol. 2014. doi:10.1111/all.12374
34. Thery C, Clayton A, Amigorena S, Raposo G. Isolation and Characterization of Exosomes from Cell Culture Supernatants and Biological Fluids. In: Current Protocols in Cell Biology; 2006.
35. Valadi H, Ekström K, Bossios A, Sjöstrand M, Lee J J, Lötvall JO. Exosome-mediated transfer of mRNAs and microRNAs is a novel mechanism of genetic exchange between cells. Nat Cell Biol. 2007. doi:10.1038/ncb1596
36. Qin J, Xu Q. Functions and applications of exosomes. Acta Pol Pharm—Drug Res. 2014. doi:10.1108/IR-11-2014-0409
37. Pisitkun T, Johnstone R, Knepper M A. Discovery of Urinary Biomarkers. Mol Cell Proteomics. 2006. doi:10.1074/mcp.r600004-mcp200
38. Mueller S K, Nocera A L, Bleier B S. Exosome function in aerodigestive mucosa. Nanomedicine Nanotechnology, Biol Med. 2018. doi:10.1016/j.nano.2017.10.008
39. Mueller S K, Nocera A L, Dillon S T, et al. Noninvasive exosomal proteomic biosignatures, including cystatin SN, peroxiredoxin-5, and glycoprotein VI, accurately predict chronic rhinosinusitis with nasal polyps. Int Forum Allergy Rhinol. 2019. doi:10.1002/alr.22226
40. Mueller S K, Nocera A L, Dillon S T, Wu D, Libermann T A, Bleier B S. Highly multiplexed proteomic analysis reveals significant tissue and exosomal coagulation pathway derangement in chronic rhinosinusitis with nasal polyps. Int Forum Allergy Rhinol. 2018. doi:10.1002/alr.22189
41. Nocera A L, Mueller S K, Stephan J R, et al. Exosome swarms eliminate airway pathogens and provide passive epithelial immunoprotection through nitric oxide. J Allergy Clin Immunol. 2018. doi:10.1016/j.jaci.2018.08.046
42. Orlandi R R, Kingdom T T, Hwang P H. International Consensus Statement on Allergy and Rhinology: Rhinosinusitis Executive Summary. Int Forum Allergy Rhinol. 2016. doi:10.1002/alr.21694
43. Nocera A L, Miyake M M, Seifert P, Han X, Bleier B S. Exosomes mediate interepithelial transfer of functional P-glycoprotein in chronic rhinosinusitis with nasal polyps. Laryngoscope. 2017. doi:10.1002/lary.26614

### Example 10. CST 1 and 2 are Associated with Multiple Post-Translational Modifications (PTMs) in CRSwNP

Samples were obtained from patients undergoing sinonasal surgery and had not been exposed to antibiotics or any topical/systemic steroids for at least 4 weeks. Inclusion criteria included patients diagnosed with CRSwNP by the International Consensus Statement on Allergy and Rhinology (ICAR:RS) criteria and healthy patients (i.e. Controls) undergoing surgery for non-inflammatory disease. Exclusion criteria included ciliary dysfunction, autoimmune disease, cystic fibrosis, or immunodeficiency. Among controls, additional exclusion criteria included the presence of allergy or asthma. Mucus samples were taken prior to tissue sampling by placing compressed polyvinyl alcohol sponges (PVA, Medtronic, Minneapolis, Minn.) against the middle meatus for 5 minutes taking care not to abrade the mucosa or contaminate the sponge with blood. Tissue and exosomes were sampled from 10 and 20 patients per group; respectively.

CST 1 and 2 were immunoprecipitated from tissue samples by adding 1.0 μg of anti-Cystatin SA antibody or anti-Cystatin SN antibody (Abcam, Cambridge, Mass.) to the tissue lysate with 20 μL of Protein A/G PLUS-Agarose (Thermofisher). After centrifugation at 3000 rpm for 5 min at 4° C., the mixture was incubated at 4° C. overnight and the immune-complex was collected by centrifugation at 3000 rpm for 5 min at 4° C. After final bead wash, samples boiled for 15 min and loaded into SDS-PAGE gel. Samples were run for 20 min at 80 V and then 120 V for 1 h. The SDS-PAGE gel was stained with Coomassie Brilliant Blue. Bands of interest were sectioned, lyophilized, and resuspended in 2-20 μL of 0.1% formic acid before LC-MS/MS analysis using Nanoflow UPLC: Easy-nLC1000 (ThermoFisher Scientific, USA) and Obitrap Q Exaxtive mass spectrometry (Thermo Fisher Scientific, USA). Raw M S files were analyzed and searched against protein sequences of the cystatin family using PEAKS (8.5, Bioinformatics Solutions, Inc). The parameters were set as follows: the protein modifications were carbamidomethylation (fixed), oxidation (variable), phosphorylation (variable), acetylation (variable), methylation (variable), deamidation (variable), dehydration (variable), Formylation (variable); the enzyme specificity was set to trypsin; the maximum missed cleavages were set to 2; the precursor ion mass tolerance was set to 10 ppm, and MS/MS tolerance was 0.05 Da. Only high confident identified peptides were chosen for downstream protein identification analysis.

Among CST1 proteins, 8 PTMs were identified at an incidence of over 50% among the CRSwNP group with no corresponding modifications in the control group. These included deamidation at N36 (75%), N40 (50%), N56 (57%), N100 (88%), and Q116 (60%); acetylation at K114 (57%), K115 (100%); and methylation at R46 (50%). Among the CST2 proteins, 6 PTMs were identified at an incidence of over 50% in the polyp group with no corresponding modifications in the control group. These included deamidation at Q24 (100%), Q76 (100%), and N82 (100%); acetylation at K114 (50%); and methylation at R28 (100%) and K96 (100%) (see FIG. 10). No significant PTMs were identified in control tissue.

These modifications could lead to a reduction in CST function with compensatory overexpression however given the variability in PTMs across all CRSwNP patients, a comprehensive analysis of the impact of each on CST function will require future study.

### BIBLIOGRAPHY

1 Wallace D V., Dykewicz M S, Bernstein D I, et al. The diagnosis and management of rhinitis: An updated practice parameter. J. Allergy Clin. Immunol. 2008; 122.
2. Smith K a, Orlandi R R, Rudmik L. Cost of adult chronic rhinosinusitis: A systematic review. Laryngoscope 2015; 1-10.
3. Rudmik L, Smith T L, Schlosser R J, Hwang P H, Mace J C, Soler Z M. Productivity costs in patients with refractory chronic rhinosinusitis. Laryngoscope 2014; 124:2007-12.
4. Soler Z M, Wittenberg E, Schlosser R J, Mace J C, Smith T L. Health state utility values in patients undergoing endoscopic sinus surgery. Laryngoscope 2011; 121:2672-8.
5. Giannoni C, Stewart M, Alford E. Intracranial complications of sinusitis. Laryngoscope 1997; 107:863-7.
6. Hedman J, Kaprio J, Poussa T, Nieminen M M. Prevalence of asthma, aspirin intolerance, nasal polyposis and chronic obstructive pulmonary disease in a population-based study. Int. J. Epidemiol. 1999; 28:717-22.
7 Klossek J M, Neukirch F, Pribil C, et al. Prevalence of nasal polyposis in France: a cross-sectional, case-control study. Allergy 2005; 60:233-7.
8. Johansson L, Akerlund A, Holmberg K, Melén I, Bende M. Prevalence of nasal polyps in adults: the Skövde population-based study. Ann. Otol. Rhinol. Laryngol. 2003; 112:625-9.
9. Gevaert P, Lang-Loidolt D, Lackner A, et al. Nasal IL-5 levels determine the response to anti-IL-5 treatment in patients with nasal polyps. J. Allergy Clin. Immunol. 2006; 118:1133-41.
10. Gevaert P, Van Bruaene N, Cattaert T, et al. Mepolizumab, a humanized anti-IL-5 mAb, as a treatment option for severe nasal polyposis. J. Allergy Clin. Immunol. 2011; 128:989-95.e1-8.
11. Wenzel S, Ford L, Pearlman D, et al. Dupilumab in persistent asthma with elevated eosinophil levels. N. Engl. J. Med. 2013; 368:2455-66.
12. Bachert C, Mannent L, Naclerio R M, et al. Effect of Subcutaneous Dupilumab on Nasal Polyp Burden in Patients With Chronic Sinusitis and Nasal Polyposis: A Randomized Clinical Trial. JAMA 2016; 315:469-79.
13. Tomassen P, Vandeplas G, Van Zele T, et al. Inflammatory endotypes of chronic rhinosinusitis based on cluster analysis of biomarkers. J. Allergy Clin. Immunol. 2016; 137:1449-1456.e4.
14. Tantilipikorn P, Bunnag C, Nan Z, Bachert C. Staphylococcus aureus superantigens and their role in eosinophilic nasal polyp disease. Asian Pacific J. Allergy Immunol. 2012; 30:171-176.
15. Zhang Z, Kofonow J M, Finkelman B S, et al. Clinical factors associated with bacterial biofilm formation in chronic rhinosinusitis. Otolaryngol. Head. Neck Surg. 2011; 144:457-462.
16. Ponikau J U, Sherris D A, Kephart G M, Adolphson C, Kita H. The role of ubiquitous airborne fungi in chronic rhinosinusitis. Clin. Rev. Allergy Immunol. 2006; 30:187-194.
17. Tan B K, Zirkle W, Chandra R K, et al. Atopic profile of patients failing medical therapy for chronic rhinosinusitis. Int. Forum Allergy Rhinol. 2011; 1:88-94.
18. Wilson K F, Mcmains K C, Orlandi R R. The association between allergy and chronic rhinosinusitis with and without nasal polyps: An evidence-based review with recommendations. Int. Forum Allergy Rhinol. 2014; 4:93-103.
19. Fokkens W J, Lund V J, Mullol J, Bachert C, Alobid I, Baroody F, Cohen N, Cervin A, Douglas R, Gevaert P, Georgalas C, Goossens H, Harvey R, Hellings P, Hopkins C, Jones N, Joos G, Kalogjera L, Kern B, Kowalski M, Price D, Riechelmann H, Schlosser R, Senior W P, Fokkens W J, Lund V J, Mullol J, Bachert C, Alobid I, Baroody F, Cohen N, Cervin A, Douglas R, Gevaert P, Georgalas C, Goossens H, Harvey R, Hellings P, Hopkins C, Jones N, Joos G, Kalogjera L, Kern B, Kowalski M, Price D, Riechelmann H, Schlosser R, Senior W P. European Position Paper on Rhinosinusitis and Nasal Polyps 2012. Rhinol Suppl. 2012 March; (23)3 p Preced. table contents, 1-298.
20. Szkudlapski D, Labuzek K, Pokora Z, et al. The emering role of helminths in treatment of the inflammatory bowel disorders. J. Physiol. Pharmacol. 2014; 65:741-751.
21. Sokol C L, Barton G M, Farr A G, Medzhitov R. A mechanism for the initiation of allergen-induced T helper type 2 responses. Nat. Immunol. 2008; 9:310-318.
22. Imoto Y, Tokunaga T, Matsumoto Y, et al. Cystatin S N upregulation in patients with seasonal allergic rhinitis. PLoS One 2013; 8:e67057.
23. Bobek L a, Levine M J. Cystatins—inhibitors of cysteine proteinases. Crit. Rev. oral Biol. Med. 1992; 3:307-332.
24. Fábián T K, Hermann P, Beck A, Fejérdy P, Fábián G. Salivary defense proteins: Their network and role in innate and acquired oral immunity. Int. J. Mol. Sci. 2012; 13:4295-4320.
25. Ganeshnarayan K, Velliyagounder K, Furgang D, Fine D H. Human salivary cystatin S A exhibits antimicrobial effect against Aggregatibacter actinomycetemcomitans. J. Periodontal Res. 2012; 47:661-673.
26. Dickinson D P. Salivary (SD-type) cystatins: Over one billion years in the making—But to what purpose? Crit. Rev. Oral Biol. Med. 2002; 13:485-508.
27. Ndika J, Airaksinen L, Suojalehto H, et al. Epithelial proteome profiling suggests the essential role of interferon-inducible proteins in patients with allergic rhinitis. J. Allergy Clin. Immunol. 2017;
28. Vilanova C, Porcar M. Are multi-omics enough? Nat. Microbiol. 2016; 1.
29. Bersanelli M, Mosca E, Remondini D, et al. Methods for the integration of multi-omics data: Mathematical aspects. BMC Bioinformatics 2016; 17.
30. Kopitar-Jerala N. The role of cystatins in cells of the immune system. FEBS Lett. 2006; 580:6295-6301.
31. Giovannini-Chami L, Marcet B, Moreilhon C, et al. Distinct epithelial gene expression phenotypes in childhood respiratory allergy. Eur. Respir. J. 2012; 39:1197-1205.
32. Poole A, Urbanek C, Eng C, et al. Dissecting childhood asthma with nasal transcriptomics distinguishes subphenotypes of disease. J. Allergy Clin. Immunol. 2014; 133.
33. Hartmann S, Lucius R. Modulation of host immune responses by nematode cystatins. In: International Journal for Parasitology. 2003: 1291-1302.
34. Schnoeller C, Rausch S, Pillai S, et al. A Helminth Immunomodulator Reduces Allergic and Inflammatory Responses by Induction of IL-10-Producing Macrophages. J. Immunol. 2008; 180:4265-4272.
35. Orlandi R R, Kingdom T T, Hwang P H, et al. International Consensus Statement on Allergy and Rhinology: Rhinosinusitis. Int. Forum Allergy Rhinol. 2016; 6 Suppl 1:S22-S209.
36. Thery C, Amigorena S, Raposo G, Clayton A. Isolation and characterization of exosomes from cell culture supernatants and biological fluids. Curr. Protoc. Cell Biol. 2006; Chapter 3: Unit 3.22.
37. Nocera A L, Miyake M M, Seifert P, Han X, Bleier B S. Exosomes mediate interepithelial transfer of functional P-glycoprotein in chronic rhinosinusitis with nasal polyps. Laryngoscope 2017;
38. Li H, Durbin R. Fast and accurate long-read alignment with Burrows-Wheeler transform. Bioinformatics 2010; 26:589-595.
39. Van der Auwera G A, Carneiro M O, Hartl C, et al. From fastQ data to high-confidence variant calls: The genome analysis toolkit best practices pipeline. Curr. Protoc. Bioinforma. 2013;
40. Wang K, Li M, Hakonarson H. ANNOVAR: Functional annotation of genetic variants from high-throughput sequencing data. Nucleic Acids Res. 2010; 38.
41. Yu Y, Triebwasser M P, Wong E K S, et al. Whole-exome sequencing identifies rare, functional CFH variants in families with macular degeneration. Hum. Mol. Genet. 2014; 23:5283-5293.
42. Wagner E K, Raychaudhuri S, Villalonga M B, et al. Mapping rare, deleterious mutations in Factor H: Association with early onset, drusen burden, and lower antigenic levels in familial AMD. Sci. Rep. 2016; 6.
43. Ng P C, Henikoff S. SIFT: Predicting amino acid changes that affect protein function. Nucleic Acids Res. 2003; 31:3812-3814.
44. Adzhubei I, Jordan D M, Sunyaev S R. Predicting functional effect of human missense mutations using PolyPhen-2. Curr. Protoc. Hum. Genet. 2013;
45. 1000 Genomes Project Consortium, Auton A, Brooks L D, et al. A global reference for human genetic variation. Nature 2015; 526:68-74.
46. Lek M, Karczewski K J, Minikel E V., et al. Analysis of protein-coding genetic variation in 60,706 humans. Nature 2016; 536:285-291.
47. Gold L, Ayers D, Bertino J, et al. Aptamer-based multiplexed proteomic technology for biomarker discovery. PLoS One 2010; 5:e15004.
48. Li C, Hung Wong W. Model-based analysis of oligonucleotide arrays: model validation, design issues and standard error application. Genome Biol. 2001; 2: Genome Biol. 2001; 2(8): research0032.1-research0032.11.
49. Benjamini Y H Y. Controlling the False Discovery Rate: A Practical and Powerful Approach to Multiple Testing. J. R. Stat. Soc. Ser. B Vol. 57, No. 1 (1995), pp. 289-300.
50. Sneath P, Sokal R. Numerical Taxonomy: The Principles and Practice of Numerical Classification. W.H. Free. Company, San Fr. 1973; 573.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or reducing risk of developing chronic rhinosinusitis with polyps in a subject, the method comprising:
identifying a subject having chronic rhinosinusitis with polyps, or who has chronic rhinosinusitis without polyps and is at risk of developing polyps; and
administering to the subject an effective amount of a cystatin inhibitor that inhibits Cystatin 1 (CST1) or CST2.

2. The method of claim 1, wherein the subject has chronic rhinosinusitis with polyps.

3. The method of claim 1, wherein the cystatin inhibitor inhibits CST2.

4. The method of claim 1, wherein the cystatin inhibitor is an antibody that binds specifically to CST1 or CST2.

5. The method of claim 1, wherein the cystatin inhibitor is an inhibitory nucleic acid that targets CST1 or CST2.

6. The method of claim 1, wherein the cystatin inhibitor decreases expression of CST1 and/or CST2 in the subject's sinonasal epithelial cells.

7. The method of claim 1, wherein the cystatin inhibitor is administered systemically.

8. The method of claim 1, wherein the cystatin inhibitor is administered locally to the subject's nasal passage and sinuses.

9. The method of claim 8, wherein the cystatin inhibitor is delivered to the subject's nasal passage and sinuses by an inhalation device, by flushing, or by spraying.

10. The method of claim 8, wherein the cystatin inhibitor is administered to the subject as a cystatin inhibitor eluting implant, stent, or spacer placed in the subject's nasal passage or sinuses, optionally implanted submucosally.

11. The method of claim 10, wherein the cystatin inhibitor eluting implant is bioabsorbable.

12. The method of claim 1, wherein the subject having rhinosinusitis was identified by endoscopy or by computed tomography.

13. The method of claim 1, wherein the subject having rhinosinusitis was identified by a method comprising:
obtaining a sample comprising nasal mucus derived exosomes, preferably a sample obtained from the posterior nasal cavity;
determining a level of one, two or all three biomarkers selected from (i) one or both of CST1 or CST2; (ii) PRDX5 (Peroxiredoxin-5); and (iii) GP6 (Platelet glycoprotein VI) in the sample;
comparing the level of the biomarker to a reference level; and
administering an effective amount of a cystatin inhibitor to a subject who has a level of any one or more of the biomarkers above the reference level.

14. The method of claim 1, wherein the subject having rhinosinusitis was identified by observing the subject's symptoms and duration of symptoms.

15. The method of claim 1, further comprising monitoring the efficacy of the treatment by endoscopy.

16. The method of claim 1, further comprising monitoring the efficacy of the treatment by computed tomography.

17. The method of claim 1, further comprising monitoring the efficacy of the treatment by observing the subject's symptoms and duration of symptoms.

18. The method of claim 1, further comprising surgically removing any nasal polyps present in the subject.

19. A kit for treating rhinosinusitis in a subject, said kit comprising
a pharmaceutical composition comprising an effective amount of a cystatin inhibitor; and
a device for delivering the pharmaceutical composition to the subject's nasal passage and sinuses.

20. The kit of claim 19, wherein said device delivers the pharmaceutical composition to the subject's nasal passage and sinuses in a liquid, nebulized, or aerosolized form.

21. The method of claim 1, wherein the cystatin inhibitor is administered in combination with one, two, or more of a corticosteroid, a decongestant, an inhibitor of inflammation, and an antibiotic.

22. The method of claim 21, wherein the corticosteroid is selected from dexamethasone, prednisone, prednisolone, triamcinolone, cortisol, budesonide, mometasone, fluticasone, flunisolide, and betamethasone.

23. The method of claim 21, wherein the antibiotic is selected from erythromycin, doxycycline, tetracycline, penicillin, beta-lactam, macrolide, fluoroquinolone, cephalosporin, and sulfonamide.

24. The method of claim 1 or 21, wherein the cystatin inhibitor is administered in combination with a corticosteroid and an antibiotic.

25. The kit of claim 19, further comprising a corticosteroid.
